(19) United States
(12) Patent Application Publication
(10) Pub. No.: US 2006/0019289 A1
(43) Pub. Date: Jan. 26, 2006

(54) Compositions and methods for gene expression analysis

(75) Inventor: Kai Qin Lao, Pleasanton, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/173,906

(22) Filed: Jun. 30, 2005

Related U.S. Application Data
(60) Provisional application No. 60/584,596, filed on Jun. 30, 2004.

(51) Int. Cl. C12Q 1/68; C12P 19/34

(52) U.S. Cl. 435/6; 435/91.2

(57) ABSTRACT

The present disclosure provides methods and composition to detect or quantitate one or more target sequences.

This application claims benefit under 35 U.S.C. § 119(e) of U.S. Patent Application Ser. No. 60/584,596, filed Jun. 30, 2004, which is incorporated herein by reference in its entirety.

### 1. FIELD

This disclosure relates generally to compositions, methods, and kits for carrying out nucleic acid sequence amplification, and more specifically to compositions, methods and kits for gene expression analysis.

### 2. INTRODUCTION

An objective of gene expression analysis is to comprehensively examine the transcriptional activity of a cell. This approach to cell analysis has identified alternatively spliced transcripts, groups of related genes, and established the order and timing of transcription regulatory mechanisms. Gene expression analysis also has been used clinically, for example, to identify classes of tumors, evaluate treatment protocols, and predict clinical outcomes. As the entire genomes of various organisms are sequenced, including the entire sequence of the human genome, gene expression analysis will play a more prominent role in medical diagnosis, treatment evaluation, and prognosis. Tens of thousands of potential genes have been identified in the human genome. Therefore, a comprehensive examination of each gene that potentially may be expressed in any cell is technically challenging. To be suitable for routine use, methods of gene expression analysis should be able to identify and quantitate expressed genes accurately, efficiently, and in a multiplex format.

The most common methods used for gene expression analysis are array based assays and quantitative polymerase chain reaction (PCR). In general, array based assays are less sensitive and less specific than quantitative PCR. However, array based assays have a very high throughput capacity, because large numbers of assays can be run simultaneously and signal detection is relatively simple. In contrast, quantitative PCR assays are more sensitive and specific than array based assays, and have a higher dynamic range. But, quantitative PCR continuously monitors product accumulation and therefore is relatively slow, requiring about two hours for completion. The reaction rate of quantitative PCR is further extended by the low throughput capacity of existing PCR machines. Furthermore, transcripts present at a very high copy number may successfully compete with low copy number transcripts, which may not be amplified to a detectable level.

There is, accordingly, a need in the art for methods of accurately identifying and quantitating differentially expressed genes, particularly in complex polynucleotide samples.

### 3. SUMMARY

Disclosed herein are compositions and methods for analyzing, e.g., detecting and/or quantitating, target polynucleotide sequences. In some embodiments, a target sequence can be amplified by forward and reverse amplification primers and a polymerase to produce double-stranded amplicons. In some embodiments, the forward and or reverse primers introduce into the amplicons sequences suitable for detecting and/or quantitating the amplicons and target sequences. In some embodiments, sequences incorporated into the amplicons can be universal sequences and/or code sequences.

In some embodiments, the amplicons are analyzed using two or more detection polynucleotides which can be modified in the presence of the amplicons. In some embodiments, detection polynucleotides comprise a detection primer and a flap probe which form a substrate for the 5′-3′ nuclease activity of a polymerase when the flap probe is hybridized to the amplicon 3′ relative to the detection primer. The nuclease activity releases the flap or cleavage sequence from the probe which may be detected or, in some embodiments, may be modified prior to detection. In some embodiments, the detection polynucleotides comprise two probes which can be ligated when hybridized to the amplicon, and the ligated product can be detected, or in some embodiments, may be further modified prior to detection.

In some embodiments, multiple sets of primers and detection polynucleotides can be used to detect or quantitate a plurality of amplicons produced from a plurality of target sequences. In some embodiments, the plurality of target sequences can be cDNA produced from reverse transcription of cellular mRNA. Therefore, in some embodiments, the methods can be used for gene expression analysis of one or more cells.

In some embodiments, control sequences are amplified to produce double-stranded control amplicons. In some embodiments, double-stranded control amplicons are suitable to provide standards for quantitating target sequences. In an alternative embodiment, the double-stranded control amplicons may be amplified and analyzed in parallel or in a multiplex format with target sequences. Therefore, in some embodiments the detection polynucleotides suitable for analyzing the control amplicons are substantially unique from the detection polynucleotides suitable for analyzing double-stranded amplicons produced from the target sequences.

In another aspect, the disclosure provides kits suitable for practicing the various embodiments of the disclosed methods. In some embodiments, a kit may comprise one or more reverse primers suitable for synthesis of double-stranded amplicons from a target sequence and/or a control sequence. In some embodiments, the kits can include one or more sets of detection polynucleotides suitable for detecting one or more of the various types of amplicons. Kits also may include one or more other reagents suitable for modifying the detection polynucleotides.

### 4. BRIEF DESCRIPTION OF THE DRAWINGS

The skilled artisan will understand that the drawings, described below, are for illustration purposes only. The drawings are not intended to limit the scope of the present disclosure in any way.

FIG. 1 provides a cartoon illustrating one embodiment of the disclosed methods. cDNA target sequences and cDNA control target sequences are amplified by PCR using forward and reverse primers. The forward primers incorporate a universal sequence and a code sequence into each amplicon. A detection primer and a detection flap probe comprising a fluorescent label (F) are hybridized to the amplicons to form a substrate for the 5′-3′ nuclease activity of a polymerase, which releases the flap sequence. The flap sequences released from the target and control sequences differ in length by at least one nucleotide (Tₙ) and are ligated to ligation partners comprising an electrophoresis mobility modifier (—O—O—). Therefore, the ligation amplicons from the sample reaction and the control reaction differ in length by at least one nucleotide and may be co-electrophoresed for detection and analysis.

FIG. 2 provides a cartoon illustrating one embodiment of the disclosed methods. cDNA target sequences and cDNA control target sequences are amplified by PCR using forward and reverse primers. The forward primers incorporate a universal sequence and a code sequence into each amplicon. A universal ligation probe (UF-LP) and an amplicon specific code ligation probe are hybridized to the amplicons. The universal forward ligation probe hybridize to the control amplicons (UF-Probe_{C}) has at least one additional nucleotides (Tₙ) in comparison to the corresponding probe hybridized to the sample amplicons (IF-Probe_{S}). Therefore, the ligation amplicons from the sample reaction and the control reaction differ in length by at least one nucleotide and may be co-electrophoresed for detection and analysis.

FIG. 3 provides the results of a multiplex gene expression analysis of liver mRNA. Ligation amplicons produced by one embodiment of the disclosed methods were analyzed by capillary electrophoresis. The results are shown as fluorescence intensity vs. migration distance. Ligation amplicons indicative of the mRNAs of the APOC2, ATP5B and COX6b genes are indicated. (see Example 1).

FIG. 4, Panels A-D provide the results of a multiplex analysis of CETP, ATP7B, BRCA1 and PEX7 cloned DNA. Panels E-H provide the results of a multiplex gene expression analysis of human reference cDNA for APOC2, PPP1CA, PEX7, ATP7A, BRCA1, ATP5B, CETP and EIF1A transcripts. (see Example 2).

FIG. 5, Panels A-J provide the results of multiplex analysis of control DNA of PEX7, ATP7A, BRCA1 and CETP. The concentration of each target sequence amplified in each reaction is shown in Example 3, Table 1.

FIG. 6 provides the results of multiplex gene expression analysis of APOC2, PPP1CA, PEX7, ATP7A, BRCA1, ATP5B, CETP and EIF1A from liver and brain cDNA. Panels A-D provide the results for liver cDNA. Panels E-H provide the results for brain cDNA.

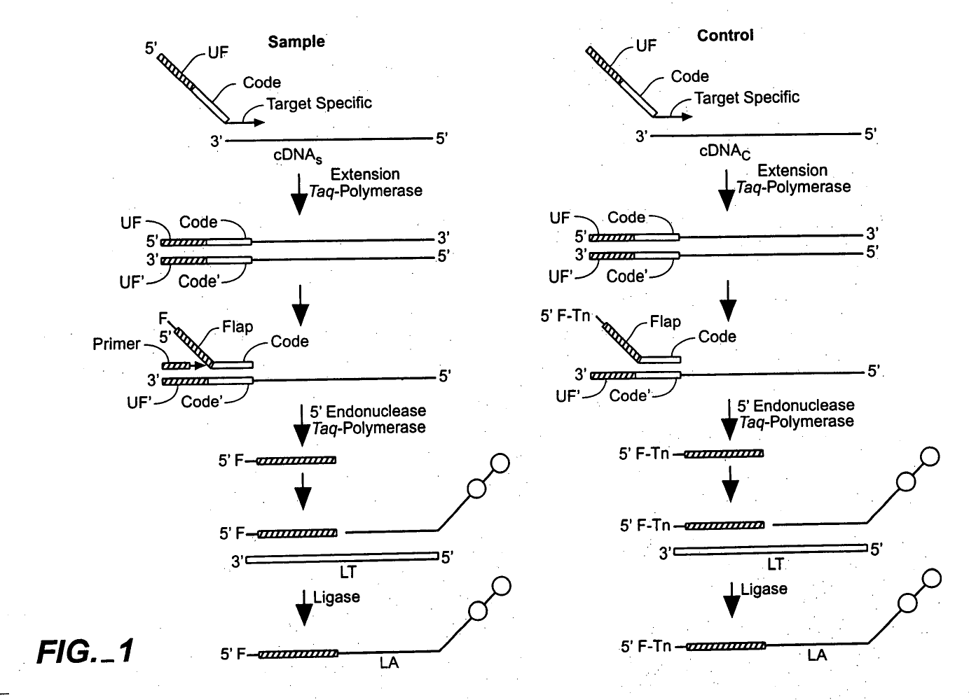

### 5. DETAILED DESCRIPTION

It is to be understood that both the foregoing general description, including the drawings, and the following detailed description are exemplary and explanatory only and are not restrictive of this disclosure. In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

This disclosure provides methods, compositions and kits for detecting and quantitating nucleic acid sequences in single-plex and multiplex formats.

As discussed in the Summary section, in some embodiments the disclosed methods comprise amplifying one or more target sequences. In some embodiments, target sequences may be amplified with a plurality of amplification primers, a polymerase, and a mixture of deoxynucleotide triphosphates (dNTPs) suitable for DNA synthesis to produce one or more amplification products (“amplicons”). In some embodiments, an amplification primer may be designed to amplify a target sequence and to introduce into the one or more amplicons one or more sequences that are utilized for downstream detection and analysis as described below. In some embodiments, the sequence introduced into the amplicon may be a code sequence which may be used as a surrogate or marker for each amplicon. In some embodiments, a sequence introduced into an amplicon may be shared by at least one other amplicon.

In some embodiments, at least two detection polynucleotides are hybridized to each amplicon. In one non-limiting example, one of the detection polynucleotides may hybridize to a sequence that is substantially unique to an amplicon. Accordingly, in various exemplary embodiments a detection polynucleotide may hybridize to a target sequence, a code sequence, or a sequence complementary thereto. When at least two detection polynucleotide are hybridized to an amplicon, at least one of the detection polynucleotides can be modified and the modified product can be detected by various methods, as described below. In some embodiments, a reporter molecule may be optionally used, for example, to monitor amplification of the target sequence and/or the modification of a detection polynucleotide. In various embodiments, the modification of a detection polynucleotide may comprise thermocycling.

In some embodiments, detection polynucleotides comprise a detection primer and a “flap” probe which form a substrate for the 5′-3′ nuclease activity of a polymerase when the flap probe is hybridized to the amplicon 3′ relative to the detection primer. The nuclease activity releases a sequence (“flap” or “cleavage” sequence) from the probe that may be detected or, in some embodiments, may be modified prior to detection. In some embodiments, the detection polynucleotides comprise two probes which can be ligated when hybridized to the amplicon, and the ligated product can be detected, or in some embodiments, may be further modified prior to detection.

As will be appreciated by skilled artisans, target polynucleotides may comprise one or more target sequences and may be either DNA (e.g., cDNA, genomic DNA or extrachromosomal DNA) or RNA (e.g., mRNA, rRNA or genomic RNA) in nature, and may be derived or obtained from virtually any sample or source, wherein the sample may optionally be scarce or of a limited quantity. For example, the sample may be one or a few cells collected from a crime scene or a small amount of tissue collected via biopsy. In other embodiments, the target polynucleotide may be a synthetic polynucleotide comprising nucleotide analogs or mimics, as described below, produced for purposes, such as, diagnosis, testing, or treatment.

In various non-limiting examples, the target polynucleotide may be single or double-stranded or a combination thereof, linear or circular, a chromosome or a gene or a portion or fragment thereof, a regulatory polynucleotide, a restriction fragment from, for example, a plasmid or chromosomal DNA, genomic DNA, mitochondrial DNA, DNA from a construct or a library of constructs (e.g., from a YAC, BAC or PAC library), RNA (e.g., mRNA, rRNA or vRNA) or a cDNA or a cDNA library. As known in the art, a cDNA is a single- or double-stranded DNA produced by reverse transcription of an RNA template. Therefore, some embodiments, in addition to the primers, probes, and enzymes, described herein, include a reverse transcriptase and one or more “RT” primers suitable for reverse transcribing an RNA template into a cDNA. Reactions, reagents and conditions for carrying out such “RT” reactions are known in the art (see, e.g., Blain et al., 1993, J. Biol. Chem. 5:23585-23592; Blain et al., 1995, J. Virol. 69:4440-4452; PCR Essential Techniques 61-63, 80-81, (Burke, ed., J. Wiley & Sons 1996); Gübler et al., 1983, Gene 25:263-269; Gübler, 1987, Methods Enzymol., 152:330-335; Okayama et al., 1982, Mol. Cell. Biol. 2:161-170; Sellner et al., 1994, J. Virol. Method. 49:47-58; and U.S. Pat. Nos. 5,310,652, 5,322,770, and 6,300,073, these disclosures of which are incorporated herein by reference.

The target polynucleotide may include a single polynucleotide, from which one or more different target sequences of interest may be analyzed, or it may include a plurality of different polynucleotides, from which one or more different target sequences of interest may be analyzed. As will be recognized by skilled artisans, the sample or target polynucleotide may also include one or more polynucleotides comprising sequences that are not analyzed by the disclosed methods.

In some embodiments, highly complex mixtures of target sequences from highly complex mixtures of polynucleotides are analyzed in either a single-plex or multiplex format. Indeed, many embodiments are suitable for multiplex analysis of target sequences from tens, hundreds, thousands, hundreds of thousands or even millions of polynucleotides. In some embodiments, multiplex amplification methods can be used to analyze pluralities of target sequences from samples comprising cDNA libraries or total mRNA isolated or derived from biological samples, such as tissues and/or cells, wherein the cDNA or, alternatively, mRNA libraries may be quite large. For example, cDNA libraries or mRNA libraries constructed from several organisms or from several different types of tissues or organs can be amplified according to the methods described herein.

As the skilled artisan will appreciate, in multiplex embodiments multiple sets of primers and/or probes and/or reporter molecules are utilized for each target sequence to be analyzed. For example, in multiplex embodiments utilizing reporter molecules, each reporter molecule can produce a signal that is distinguishable from other reporter molecules. Therefore, in these embodiments, the number of target sequences analyzed in a multiplex format can be determined, at least in part, by the number and type of reporter molecules that may be discriminated. For example, in the embodiment, in which 5′-nuclease are probes are utilized as the reporter molecule about 2 to about 7 target sequences are analyzed in a multiplex reaction. However, in other embodiments in which detection polynucleotides described herein are utilized about 2 to about 1,000 target sequences and in some embodiments to about 7,000 target sequences or more can be analyzed in a multiplex reaction. (see, e.g., U.S. Patent Application Ser. Nos. 60/584,621; 60/584,665; 60/584,643, each filed Jun. 30, 2004).

The amount of target polynucleotide(s) utilized in the disclosed methods can vary widely. In many embodiments, amounts suitable for a conventional PCR and/or RT-PCR may be used. For example, the target polynucleotide(s) may be from a single cell, from tens of cells, from hundreds of cells or even more, as is well known in the art. For many embodiments, including embodiments in which the target polynucleotide is a complex cDNA library (or derived therefrom by RT of mRNA), the total amount of target polynucleotide utilized may range from about 1 pg to about 100 ng. For some embodiments, including embodiments in which the target polynucleotide(s) is obtained from a single cell, the total amount of target polynucleotide(s) may range from 1 copy (about 10 ag) to about 10⁷ copies (about 100 pg). In some embodiments target polynucleotides may range from about 100 to about 10⁶ copies. The skilled artisan will appreciate that in various embodiments a greater number of target polynucleotides may be used or the number of target polynucleotides is unknown.

In some embodiments, preparation of the target polynucleotide(s) for analysis may not be required. In some embodiments, the target polynucleotide(s) may be prepared for analysis using conventional sample preparation techniques. For example, target polynucleotides may be isolated from their source via chromatography, precipitation, electrophoresis, as is well-known in the art. Alternatively, the target sequence(s) may be amplified directly from samples, including but not limited to, cells or from lysates of tissues or cells comprising the target polynucleotide(s). Therefore, as used herein, “target sequence” also refers to an amplified target sequence. Furthermore, in some embodiments, a target sequence may be amplified but multiple sets of primers. Examples of suitable amplification methods are well known in the art (see, e.g., U.S. Pat. Nos. 4,683,195, 4,683,202, 4,800,159, 4,965,188, 5,075,216, 5,176,995, 5,185,243, 5,386,022, 5,427,930, 5,516,663, 5,656,493, 5,679,524, 5,686,272, 5,869,252, 6,040,166, 6,197,563, 6,514,736, EP-A-0200362, EP-A-0201184 and EP-A-320308, and U.S. Patent Application Ser. No. 60/584,665, filed Jun. 30, 2004). Therefore, in some embodiments a target sequence may be amplified by one or more amplification primers to produce amplicons that may be further amplified by the disclosed methods.

As will be appreciated by skilled artisans, control polynucleotides may comprise one or more control sequences and may be of any composition or source, or may be prepared or utilized at concentrations as described above for target polynucleotides. In some embodiments, one or more control polynucleotides may be amplified and analyzed by the disclosed methods to ensure the proper function of the reaction conditions or reagents. In some embodiments, a control polynucleotide may be amplified and analyzed to provide standards from which one or more target sequences may be quantitated. Therefore, in some embodiments, one or more control polynucleotides are quantitated prior to analysis by the disclosed methods, as described below. In some embodiments, a control polynucleotide may be substantially identical to a target polynucleotide. However, the skilled artisan will appreciate that in some embodiments substantial identity between a target and control polynucleotide may not be required.

The number of target sequences that can be analyzed by the disclosed methods is influenced in large part by the number of different amplification primers, detection polynucleotides, and the number of different methods used to detect or discriminate the modified detection polynucleotides. In various exemplary embodiments, at least one amplification primer, at least two amplification primers, or at least three amplification primers or more may be used to amplify a target sequence. By “primer” herein is meant a polynucleotide capable of hybridizing or annealing to a template polynucleotide to form a substrate for a polymerase (e.g., DNA-dependent DNA polymerases, RNA-dependent DNA polymerase (reverse transcriptase)). When a primer is hybridized to its template, a polymerase is capable of initiating synthesis of a nascent polynucleotide strand in a template directed manner at the 3′ terminus of the primer. Therefore, in various embodiments, a primer can be an amplification primer and/or a reverse transcription primer. In some embodiments, a primer may be a detection polynucleotide, as described below. By “annealing” or “hybridizing” is meant base-pairing interactions of one nucleobase polymer with another that results in the formation of a double-stranded structure. In some embodiments, annealing occurs via Watson-Crick base-pairing interactions, but may be mediated by other hydrogen-bonding interactions, such as Hoogsteen base pairing.

In various embodiments, an amplification primer may be an “exponential primer” and/or a “linear primer.” By “exponential primer” and “exponential amplification primer” herein are meant a primer suitable for exponential amplification of a polynucleotide sequence. In exponential target sequence amplification, the product of each amplification cycle is an amplicon that is a suitable template for subsequent amplification cycles. Therefore, as known in the art, exponential amplification generally utilizes at least two or paired exponential primers. For example, the exponential amplification of a target sequence by PCR generally utilizes a pair of “forward” and “reverse” primers. Therefore, the skilled artisan is aware that the suitability of a primer for exponential amplification depends, in part, on the presence of a second suitable primer. The forward and reverse primers hybridize to a target sequence in opposite orientations to produce complementary DNA strands to form double-stranded amplicons that serve as templates for further rounds of amplification. By “linear primer” and “linear amplification primer” herein are meant a primer suitable to linearly amplify a polynucleotide sequence. In linear target sequence amplification, the product of each amplification cycle is not suitable for subsequent amplification cycles. For example, the linear amplification of a target sequence generally produces a single-stranded amplicon that does not hybridize to the linear primer and, therefore, is not a suitable template for subsequent amplification cycles. As a result, in some embodiments, linear amplicons accumulate at a rate proportional to the number of templates. Methods employing exponential and linear amplification reactions to quantitate target polynucleotides are disclosed in U.S. Patent Application Ser. No. 60/584,665, filed Jun. 30, 2004.

The amplification primers may be target sequence-specific or may be designed to hybridize to sequences that flank a target sequence to be amplified. Thus, the actual nucleotide sequences of each primer may depend upon the target sequence and target polynucleotide, which will be apparent to those of skill in the art. Methods for designing primers suitable for amplifying target sequences of interest are well-known (see, e.g., Dieffenbach et al., General Concepts for PCR Primer Design, in PCR Primer, A Laboratory Manual, Dieffenbach, C. W, and Dveksler, G. S., Ed., Cold Spring Harbor Laboratory Press, New York, 1995, 133-155; Innis, M. A. et al. Optimization of PCRs, in PCR protocols, A Guide to Methods and Applications, Innis, M. A., Gelfand, D. H., Sninsky, J. J., and White, T. J., Ed., CRC Press, London, 1994, 5-11; Sharrocks, et al. The design of primers for PCR, in PCR Technology, Current Innovations, Griffin, H. G., and Griffin, A. M, Ed., CRC Press, London, 1994, 5-11; Suggs et al., Using Purified Genes, in ICN-UCLA Symp. Developmental Biology, Vol. 23, Brown, D. D. Ed., Academic Press, New York, 1981, 683; Kwok et al. Effects of primer-template mismatches on the polymerase chain reaction: Human Immunodeficiency Virus 1 model studies. Nucleic Acids Res. 18:999-1005, 1990; Compton T (1990). Degenerate primers for DNA amplification. pp. 39-45 in: PCR Protocols (Innis, Gelfand, Sninsky and White, eds.); Academic Press, NY; Fuqua et a. (1990). BioTechniques 9(2):206-21 1; Gelfand et al., 1990, Thermostable DNA polymerases. pp. 129-141 in: PCR Protocols (Innis, Gelfand, Sninsky and White, eds.); Academic Press, NY; Innis et al., 1990, Optimization of PCRs. pp. 3-12 in: PCR Protocols (Innis, Gelfand, Sninsky and White, eds.); Academic Press, NY; Krawetz et al., 1989, Nucleic Acids Research 17(2):819; Rybicki et al., 1990, Journal of General Virology 71:2519-2526; Rychlik et al., 1990, Nucleic Acids Research 18(21):6409-6412; Sarkar et al., 1990, Nucleic Acids Research 18(24):7465; Smith et al., 1990, 9/90(5):16-17; Thweatt et al. 1990, Analytical Biochemistry 190:314-316; Wu et al., 1991, DNA and Cell Biology 10(3):233-238; Yap etal., 1991, Nucleic Acids Research 19(7): 1713, which provide examples demonstrating how particular primer pairs may be designed.

Generally, each amplification primer should be sufficiently long to prime template-directed synthesis under the conditions of the disclosed methods. The exact lengths of the primers may depend on many factors, including but not limited to, the desired hybridization temperature between the primers and template polynucleotides, the complexity of the different target polynucleotide sequences to be amplified, the salt concentration, ionic strength, pH and other buffer conditions, and the sequences of the primers and target polynucleotides. The ability to select lengths and sequences of primers suitable for particular applications is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1 -10.10 (3d. ed. Cold Spring Harbor Laboratory Press)). In some embodiments, the primers contain from about 15 to about 35 nucleotides that are suitable for hybridizing to a target sequence and form a substrate suitable for DNA synthesis, although the primers may contain more or fewer nucleotides. Shorter primers generally require lower temperatures to form sufficiently stable hybrid complexes with target sequences. The capability of polynucleotides to anneal can be determined by the melting temperature (“Tₘ”) of the hybrid complex. Tₘ is the temperature at which 50% of a polynucleotide strand and its perfect complement form a double-stranded polynucleotide. Therefore, the Tₘ for a selected polynucleotide varies with factors that influence or affect hybridization. In some embodiments, in which thermocycling occurs, the amplification primers should be designed to have a melting temperature (“Tₘ”) in the range of about 60-75° C. Melting temperatures in this range tend to insure that the primers remain annealed or hybridized to the target polynucleotide at the initiation of primer extension. The actual temperature used for a primer extension reaction may depend upon, among other factors, the concentration of the various primers and the types of detection polynucleotides employed, as described below, and methods used to detect the modified detection polynucleotides. For amplifications carried out with a thermostable polymerase such as Taq DNA polymerase, the amplification primers can be designed to have a Tₘ in the range of about 60 to about 78° C. The melting temperatures of the different amplification primers can be different; however, in an alternative embodiment they should all be approximately the same, i.e., the Tₘ of each amplification primer can be within a range of about 5° C. or less. The Tₘs of various primers can be determined empirically utilizing melting techniques that are well-known in the art (see, e.g., Sambrook et al. Molecular Cloning: A Laboratory Manual 11.55-11.57 (2d. ed., Cold Spring Harbor Laboratory Press)).

Alternatively, the Tₘ of a amplification primer can be calculated. Numerous references and aids for calculating Tₘs of primers are available in the art and include, by way of example and not limitation, Baldino et al. Methods Enzymology. 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res. 18:6409-6412 (erratum, 1991, Nucleic Acids Res. 19:698); Rychlik. J. NIH Res. 6:78; Sambrook et al. Molecular Cloning: A Laboratory Manual 9.50-9.51, 11.46-11.49 (2d. ed., Cold Spring Harbor Laboratory Press); Sambrook et al., Molecular Cloning: A Laboratory Manual 10.1-10.10 (3d. ed. Cold Spring Harbor Laboratory Press)); Suggs et al., 1981, In Developmental Biology Using Purified Genes (Brown et al., eds.), pp. 683-693, Academic Press; Wetmur, 1991, Crit. Rev. Biochem. Mol. Biol. 26:227-259, which disclosures are incorporated by reference. Any of these methods can be used to determine a Tₘ of a primer.

As the skilled artisan will appreciate, in general, the relative stability and therefore, the Tₘs, of RNA:RNA, RNA:DNA, and DNA:DNA hybrids having identical sequences for each strand may differ. In general, RNA:RNA hybrids are the most stable (highest relative Tₘ) and DNA:DNA hybrids are the least stable (lowest relative Tₘ). Accordingly, in some embodiments, another factor to consider, in addition to those described above, when designing any primer is the structure of the primer and target polynucleotide. For example, in the embodiment in which an RNA polynucleotide is reverse transcribed to produce a cDNA, the determination of the suitability of a DNA primer for the reverse transcription reaction should include the effect of the RNA polynucleotide on the Tₘ of the primer. Although the Tₘs of various hybrids may be determined empirically, as described above, examples of methods of calculating the Tₘ of various hybrids are found at Sambrook et al. Molecular Cloning: A Laboratory Manual 9.51 (2d. ed., Cold Spring Harbor Laboratory Press).

The concentration of an amplification primer may vary widely and in various embodiments, may be limiting or non-limiting. “Limiting concentration” refers to a concentration of a reagent, such as, an amplification primer, that determines the rate at which a reaction may proceed and/or the time point at which a reaction terminates. Conversely, “non-limiting concentration” refers to a concentration of a reagent at the point a reaction initiates that may not determine the rate at which the reaction may proceed and/or the time point at which the reaction terminates. A skilled artisan will appreciate, however, that in some embodiments a reagent at a non-limiting concentration may become limiting as the reagent is consumed during the course of the reaction. In some embodiments, a limiting concentration of an amplification primer terminates the amplification reaction before it reaches a plateau. In some embodiments, the concentration of an amplification primer can be adjusted so that a selected number of amplicons are generated. Determining the appropriate concentration of one or more amplification primers is within the abilities of the skilled artisan. Examples of factors to be considered include but are not limited to the quantity of target sequence, the relative amount of each target polynucleotide sequence to be amplified, the number of different target polynucleotides sequences amplified in a single reaction (i.e., multiplex or single-plex), the sensitivity of the detection system, and the degree of accuracy desired. In various exemplary embodiments, a limiting concentration of an amplification primer is less than about 50 nM, less than about 40 nM, less than about 30 nM, less than about 20 nM, or less than about 10 nM. In some embodiment, a limiting concentration of an amplification limiting primer is about 10 nM to about 30 nM. Exemplary embodiments of non-limiting concentrations include, a concentration of at least about 100 nM, at least about 500 nM, at least about 1 μM or even greater. 1000381 The target specific sequences of amplification primers used in the disclosed methods are designed to be substantially complementary to regions of the target polynucleotides. By “substantially complementary” herein is meant that the sequences of the amplification primers include enough complementarity to hybridize to the target polynucleotides at the concentration and under the temperature and conditions employed and to be capable of being extended by a DNA polymerase. Although in some embodiments the sequences of the primers may be completely complementary to a target polynucleotide, in other embodiments it may be desirable to include one or more nucleotides of mismatch or non-complementarity, as is well known in the art. By “regions of mismatch” and “non-complementarity” are meant a least one nucleotide of a polynucleotide sequence that is not suitable for base-pairing with another polynucleotide sequence. Therefore, the term “region of mismatch” is used when comparing sequences, such as, a primer sequence and another primer sequence; a primer sequence and a target sequence; a probe sequence and a target sequence; a primer sequence and an amplicon sequence; and the like. Therefore, a “region of mismatch” includes a “region of sequence diversity.” As the skilled artisan will appreciation, a region of mismatch between an amplification primer and a target sequence may be incorporated into the resulting amplicons. In some embodiments, regions of mismatch may be incorporated into amplicons to provide useful cites for hybridizing to detection polynucleotides, as described below.

In some embodiments, an amplification primer sequence that is a region of mismatch in comparison to a target sequence is substantially unique to that primer. Therefore, in some embodiments, a region of mismatch between an amplification primer and a target sequence is a code sequence. By “code sequence” is meant a sequence of continuous nucleotides that is substantially unique. “Substantially unique” refers to a sequence suitable to identify or distinguish the polynucleotide comprising the code sequence. In some embodiments, code sequences may be used to identify the amplification product of a specific primer and/or to identify the product of a modified detection polynucleotide. Therefore, the skilled artisan will appreciate that in some embodiments code sequences may be used for the manipulation, detection and/or analysis of polynucleotides and accordingly may be used in sequences of primers, probes, templates and the like.

In some embodiments, a region of mismatch between an amplification primer and a target sequence is a sequence that is shared by more than one amplification primer. In some embodiments, the shared sequence may also be a sequence of a probe. In non-limiting exemplary embodiments, a “shared sequence” may be common to each forward primer or each reverse primer. Thus, “forward universal sequence” and “reverse universal sequence” refer to a primer sequence of continuous nucleotides that is a region of diversity in comparison to a target sequence that is shared by each forward or reverse primer, respectively.

Determining the number, type, length, and composition of the various regions of an amplification primer and their distribution or commonality among the various polynucleotides employed in the disclosed methods are within the capabilities of the ordinary skilled artisan. Amplification primers and methods for incorporating various types of sequences into amplification primers and amplicons derived therefrom are known in the art (see, e.g., U.S. Pat. Nos. 5,314,809, 5,853,989, 5,882,856, 6,090,552, 6,355,431, 6,617,138, 6,630,329, 6,635,419, 6,670,130, 6,670,161 and Weighardt et al., 1993, PCR Methods and App. 3:77 and, the disclosures of which are incorporated by reference).

To detect and analyze the one or more amplicons produced by the disclosed methods, in some embodiments, the amplicons are hybridized to at least two detection polynucleotides. When hybridized to the amplicon, the detection polynucleotides form a substrate which is modified to form a detectable product. In various exemplary embodiments, “modified” refers to cleavage, extension, ligation, and/or labeling of a detection polynucleotide. The skilled artisan will appreciate that in some embodiments, including but not limited to multiplex reactions, each pair of detection polynucleotides may comprise a substantially unique substrate which is modified to form a substantially unique product. Therefore, each product detected may be traced to a specific amplicon and target sequence.

In some embodiments, the detection polynucleotides comprise a “flap” probe and a detection primer. “Flap probe” or “cleavage probe” refers to a probe comprising at least two domains or regions. One probe domain comprises a nucleobase sequence suitable for hybridizing to a target polynucleotide and, therefore, is substantially complementary to a target sequence. Another probe domain comprises a nucleobase sequence that is not suitable for hybridizing to a target polynucleotide. Therefore, when a flap probe hybridizes to its target polynucleotide, one domain of the probe forms one strand of a double-stranded nucleic acid and another domain forms a single-stranded region, i.e., a “flap” or “cleavage” sequence. The skilled artisan will appreciate that the definition of flap probe provided herein, differs from a “conventional probe” which does not provide a “flap” or “cleavage” sequence suitable for release by the 5′-3′ activity of a polymerase when hybridized to its complementary sequence, and wherein the released flap or cleavage sequence is suitable for detection as described below. In various embodiments, the target specific and flap sequences may be in any orientation. Therefore, in some embodiments, the flap sequence is 5′ relative to the target specific sequence, and, in some embodiments, the flap is 3′ relative to the target specific sequence.

In some embodiments, thermocycling is employed to form additional substrates for the nuclease activity of the polymerase. In some embodiments, a substrate for the 5′-3′ nuclease activity may be formed by the hybridization of the flap probe and detection under conditions suitable for extension of the detection primer by the polymerase.

In various exemplary embodiments, the target specific sequences of the flap probes may be designed to be substantially complementary to the target sequence, to a region of the amplicon that flanks the target sequence, including but not limited to, a universal sequence, a code sequence, or sequences complementary thereto. The actual nucleobases that comprise each hybridization sequence may depend upon the complexity of the target polynucleotides being analyzed, and the number of type of sequences incorporated into the amplicons, which will be apparent to those of skill in the art. Generally, the parameters described above in the design of amplification primers are applicable to the design of the target specific sequences of the flap probes.

In contrast to the target specific sequences, the flap or cleavage sequences of the probes are designed to be substantially non-complementary to the target polynucleotides. Therefore, the flap sequences are regions of mismatch relative to the target polynucleotides. The actual nucleobases that comprise each flap sequence may depend upon the number and type of target sequences and target polynucleotides to be analyzed, the assay conditions (e.g., temperature, pH, ionic strength, etc.), and the extent to which each flap sequence may be discriminated. Therefore, in some embodiments, each flap sequence may be substantially unique or provide a code sequence. In some embodiments, a flap sequence may not be substantially unique and, therefore, may have statistically significant sequence homology to the flap sequence of another flap probe. Therefore, in some embodiments, two or more flap sequences may be identical in length and/or composition. Embodiments in which such flap sequences find use include, but are not limited to, assays in which a sample is screened for the presence or absence of one or more target polynucleotides. In such embodiments, discrimination of the released flap sequences and, therefore, discrimination of the various target polynucleotides is generally not desired.

In some embodiments, wherein the flap sequence is released by the 5′-3′ nuclease activity of a polymerase, the probe is hybridized to a target sequence 3′ relative to a detection primer. By “detection primer” herein is meant a polynucleotide capable of hybridizing or annealing to a template polynucleotide to form a substrate for a polymerase at a position that is 5′ relative to a flap probe. Therefore, when a detection primer is hybridized to a target polynucleotide at a position 5′ relative to a flap probe, a substrate for the 5′-3′ nuclease activity of a polymerase is formed. In some embodiments, a detection primer may also function as an amplification primer as described above. In some embodiments, a detection primer and an amplification primer are different polynucleotides. Therefore, in some embodiments, a detection primer may hybridize to a universal sequence, a code sequence or sequences complementary thereto. Generally, the parameters described above in the design of amplification primers are applicable to the design of the detection primers.

Non-limiting examples of polymerases with 5′-3′ nuclease activity that may be suitable for release of the flap sequence include, but are not limited to, AmpliTaq® GOLD, AmpliTaq® FS and AmpliTaq® DNA polymerase (Applied Biosystems, Foster City, Calif.), E. coli DNA polymerase I (New England Biolabs, Beverly, Mass.), rBst DNA Polymerase (Epicenter®, Madison, Wis.), and Tf1 DNA polymerase (Promega Corp., Madison, Wis.). The nuclease activity of the polymerase and its capability to release the flap sequence is, at least in part, influenced by the distance between the 3′ terminus of the detection primer and the most 5′ nucleobase of the probe that is hybridized to the target sequence. Therefore, in some embodiments, extension of the primer by the polymerase may not be required for the flap probe to be released. For example, if the 3′ terminus of the primer is at least within about 20 nucleobases of the 5′ hybridized nucleobase of the probe, primer extension and, therefore, amplification may not be required for release of the flap sequence. In embodiments wherein the distance between the primer and probe is greater than about 20 nucleobases, extension of the detection primer may be required for release of the flap sequence from a probe. Therefore, in some embodiments, the primer may be extended such that its 3′ terminus is within at least about 20 nucleobases of the hybridized probe. In some embodiments, the primer may be extended to amplify the target sequence. Therefore, in some embodiments, release of the flap sequence may occur during amplification of the target sequence, with or without thermocycling.

Once released, the flap sequences may be detected or quantitated by various techniques as known in the art. In some embodiments, a released flap sequence may be detected or quantitated directly without modification. Therefore, in some embodiments the released flap sequence is detected or quantitated in the form in which is it released. In various non-limiting examples, a released flap sequence may be directly detected or quantitated by capillary electrophoresis (see, e.g., U.S. Pat. Nos. RE37,941, 6,372,106, 6,372,484, 6,387,234, 6,387,236, 6,402,918, 6,402,919, 6,432,651, 6,462,816, 6,475,361, 6,476,118, 6,485,626, 6,531,041, 6,544,396, 6,576,105, 6,592,733, 6,596,140, 6,613,212, 6,635,164, 6,706,162) or by array-based assays (see, e.g., U.S. Pat. Nos. 5,405,783, 5,445,934, 5,510,270, 5,547,839, 6,232,062, 6,221,583, 6,309,822, 6,344,316, 6,355,431, 6,355,432, 6,368,799, 6,396,995, 6,410,229, 6,440,667, 6,576,425, 6,576,424, 6,600,031, 6,632,605, 6,646,243, 6,495,323, 6,667,394, 6,670,122, 6,686,150). However, the skilled artisan will appreciate that, in some embodiments, a modified flap sequence, as described below, may be detected or quantitated by methods suitable for use with an unmodified flap sequence.

In some embodiments, a released flap sequence may be modified. For example, in some embodiments, the released flap sequence may comprise the ligand of a binding partner or an anti-ligand. Therefore, in some embodiments, a flap sequence is modified by the binding of the binding partner to the ligand. Thus, “ligand,” “binding partner” and “anti-ligand” as used herein refer to molecules that specifically interact with each other. “Specifically interact” refers to binding that is substantially distinctive and restricted, and sufficient to be sustained under conditions that inhibit non-specific binding. Non-limiting examples of ligand binding include but are not limited to antigen-antibody binding (including single-chain antibodies and antibody fragments (e.g. Fab, Fab′, F(ab′)₂, Fv)), hormone-receptor binding, neurotransmitter-receptor binding, polymerase-promoter binding, substrate-enzyme binding, allosteric effector-enzyme binding, biotin-streptavidin binding, digoxin-anti-digoxin binding, carbohydrate-lectin binding, or a molecule that donates or accepts a pair of electrons to form a coordinate covalent bond with the central metal atom of a coordination complex. In various exemplary embodiments, the dissociation constant of the ligand/anti-ligand complex is less than about 10⁻⁴-10⁻⁹ M⁻¹, less than about 10⁻⁵-10⁻⁹ M⁻¹ or less than about 10⁻⁷-10⁻⁹ M⁻¹. In some embodiments, a ligand and/or binding partner comprise one or more detectable moieties, described below.

In some embodiments, a released flap sequence is modified by the action of one or more enzymes. Non-limiting examples of enzymes suitable for modifying a released flap sequence include polymerases (e.g., DNA-directed DNA polymerases, RNA-directed DNA polymerases, terminal transferases, thermostable polymerases (e.g., Taq, Pfu, Vent), reverse transcriptases, Klenow fragment, T4 DNA polymerase, T7 DNA polymerase, ligases (e.g., thermostable ligases, T4 DNA ligase), polynucleotide kinases, phosphatases (e.g., bacterial alkaline phosphatase, calf intestinal alkaline phosphatase, shrimp alkaline phosphatase), endonucleases (e.g., restriction endonucleases I-III), and exonucleases (e.g., exonucleases I-III, mung bean nuclease, BAL31 nuclease, S1 nuclease). Therefore, in various exemplary embodiments, a released flap sequence may be modified by the addition or removal of nucleotides or phosphate groups, by ligation to another polynucleotide, by cleavage of the flap sequence, by the addition or removal of a moiety (e.g., a ligand or a moiety suitable for producing a detectable signal, as described below), or by amplification of the released flap sequence (e.g., by PCR, LCR, LDR, OLA). In some embodiments, a released flap sequence may be suitable to initiate a coupled amplification reaction (see, e.g., U.S. Patent Application Ser. No. 60/584,665, filed Jun. 30, 2004).

The skilled artisan will appreciate that in some embodiments modification, detection or quantitation of a released flap sequence may include hybridizing the released flap sequence to another polynucleotide, such as, a primer, a probe, or template (e.g., a polymerization template or ligation template). When hybridized to another polynucleotide, the released flap sequence, in various embodiments, may itself function as a probe, template, primer and/or a substrate (e.g., a ligation partner, as described below). Therefore, in some embodiments, a flap sequence may be designed to be substantially complementary to a polynucleotide that is used in methods of detecting the released flap sequence. In some embodiments, wherein a released flap sequence is ligated to a ligation partner, the flap sequence and ligation partner are hybridized to a ligation template under conditions suitable for a ligase to form a covalent bond between the 3′-hydroxyl of one polynucleotide and the 5′-phosphate of the other. Thus, in some embodiments, the resulting “ligation product” may be formed by ligating the flap sequence to the 3′ or 5′ terminus of the ligation partner. In some embodiments, the conditions suitable for ligation may include thermocycling in the presence of a thermostable ligase. In some embodiments, the flap sequence and ligation partner, when hybridized to the ligation template, may be separated by a gap of at least one nucleotide and, therefore, are not suitable for ligation. Therefore, in some embodiments, the sequence hybridized to the ligation template 5′ relative to the other sequence may be extended by the action of a polymerase. In some embodiments, a gap between the hybridized flap sequence and ligation partner may be filled-in by hybridizing one or more ligation partners to the ligation template.

Generally, each released flap sequence should be sufficiently long and comprise a sequence sufficient for its detection or quantitation by the method selected by the practitioner. Similarly, the polynucleotides employed to detect or quantitate a released flap sequence also should be sufficiently long and comprise a sequence suitable for detecting or modifying the released flap sequence. Factors to be considered in selecting the length and composition of a flap sequence and the polynucleotides employed in its detection or modification include but are not limited to, the method of detection, the efficiency of a reaction selected to modify the released flap sequence, the number of types of polynucleotides employed to detect the released flap sequences, the conditions under which the flap sequence is released, the presence or absence of moieties on the released flap sequence (e.g., ligands or detectable moieties), the complexity of the different target polynucleotides to be analyzed, the complexity of the different flap sequences, and the reaction conditions (e.g., temperature, salt concentration, ionic strength, pH, and the like). The ability to design flap sequences and polynucleotides of suitable length and composition for their detection is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1-10. 10 (3d. ed. Cold Spring Harbor Laboratory Press)). However, generally, flap sequences comprise from about 15 to about 35 nucleotides, although in some embodiments the flap sequences may contain more or fewer nucleotides/nucleobases. Furthermore, polynucleotides employed to detect or modify a released flap sequence may be shorter or longer than the flap sequence. As described above, the capability of sequences to anneal can be determined by the melting temperature (“Tₘ”) of the hybrid complex. Thus, the factors described herein in the design of target specific sequences suitable for hybridizing to a target polynucleotide are, in some embodiments, applicable to the design of flap sequences and polynucleotides used for their detection or modification.

In some embodiments, the detection polynucleotides comprise at least two ligation probes. Therefore, in some embodiments, the ligation probes hybridize to a target sequence, e.g., an amplicon, and are modified by being joined to form a single polynucleotide (“ligation amplicon”). In some embodiments, at least one ligation probe hybridizes to a sequence that is substantially unique to the amplicon. Therefore, the ligation amplicon that is produced may be traced to the amplicon and the target sequence that was amplified. In various exemplary embodiments, the substantially unique sequence may be the target sequence, a code sequence, or sequences complementary thereto. Therefore, any one or more other ligation probes may be hybridized to a substantially unique amplicon sequence or a sequence shared by other amplicons. In some embodiments a first ligation probe hybridizes to a substantially unique sequence, e.g., a code sequence and at least one other ligation probe hybridizes to a sequence shared with at least one other amplicon, e.g., a universal sequence (e.g., “universal primer”, “universal ligation probe”).

In some embodiments, the ligation probes hybridize to an amplicon to form a substrate for a ligase under conditions suitable for a ligase to form a covalent bond between the 3′ hydroxyl of one ligation probe and the 5′ phosphate of another ligation probe. In some embodiments, the conditions suitable for ligation may include thermocycling in the presence of a thermostable ligase. In some embodiments, one or more ligation probes, when hybridized to the amplicon, may be separated by a gap of at least one nucleotide and, therefore, are not suitable for ligation. Therefore, as described above for the released flap sequences, in some embodiments, the sequences hybridized to the ligation template 5′ relative to the other sequence may be extended by the action of a polymerase. In some embodiments, a gap between the hybridized flap sequence and ligation partner may be filled-in using one or more additional ligation probes. The skilled artisan will appreciate, that the ligation amplicon may be detected by any one or more of the methods described above for the released flap sequences.

In some embodiments, one or more modified detection polynucleotides are quantitated by comparison to modified detection polynucleotides obtained from the analysis of control target sequences. Therefore, “control target sequences” and “control sequences” refer to polynucleotides analyzed by the disclosed methods to provide a comparison group, which, in some embodiments, may be used to quantitate one or more target sequences. Therefore, in some embodiments the copy number or quantity of a control sequence may be determined prior to amplification and analysis by the disclosed methods (see, e.g., Sambrook et al., Molecular Cloning: A Laboratory Manual 5.5-5.17, 5.71-5.82, 6.11-6.15 (3d. ed. Cold Spring Harbor Laboratory Press)).

In some embodiments, the modified detection polynucleotides produced by the analyses of one or more control sequences (e.g., “control ligation probes”, “control detection primers,” “control flap probes” and the like) may be used to establish standards from which the quantity of one or more target sequences may be determined. In some embodiments, the modified detection polynucleotides produced by the analysis of the control and target sequences may be simultaneously analyzed. Therefore, in some embodiments, each modified detection polynucleotide produced from the analysis of a control sequence can be distinguishable or is substantially unique in comparison to each modified detection polynucleotide produced from the analysis of each target sequence. Determining the number and types of parameters suitable to differentiate modified detection polynucleotides is within the abilities of the skilled artisan. However, in one non-limiting example, a released flap sequence from the analysis of a control sequence may be at least one nucleotide longer or shorter than the released flap sequence from the analysis of the corresponding target sequence. Therefore, in some embodiments, the released sequences may be simultaneously analyzed and quantitated by capillary electrophoresis.

In some embodiments, the control and target sequences may be analyzed in parallel, separate reactions. In an alternative embodiment, the control and target sequences may be simultaneously analyzed, for example, in a multiplex reaction. Multiplex analysis of control and target sequences may be accomplished, for example, using a set of substantially unique control sequences and control detection polynucleotides. Therefore, in this non-limiting example, control sequences and their detection polynucleotides may not share substantial sequence homology with the target sequences and the target sequences′ detection polynucleotides. In some embodiments, the control sequences also may have various copy numbers to encompass the possible copy number for any given target sequence. Therefore, in some embodiments the control target sequences may vary from about 1 copy to about 10⁸ copies.

In some embodiments, the target sequences may be quantitated by log-linear amplification. Therefore, in some embodiments, the exponential amplification of the target sequences may terminate when a selected number of double-stranded amplicons are produced. The detection polynucleotides may be used to linearly amplify the double-stranded amplicons to produce linear amplicons and a detectable signal, e.g., a modified detection polynucleotide, proportional to the number of linear amplicons. As described in U.S. Patent Application Ser. No. 60/584,665, filed Jun. 30, 2004, measurements of the detectable signal may be used to calculate the copy number of the target sequence.

In the exemplary embodiments, described above, the two detection polynucleotides are hybridized to one strand of a amplicon. Thus, in some embodiments the detection polynucleotides may be employed in a linear amplification reaction. The skilled artisan will appreciate that in some embodiments the detection polynucleotides may be employed in an exponential amplification reaction. In one non-limiting example, the forward and reverse primers for the production of a double-stranded amplicon each may incorporate a code sequence and universal sequence into both strands of the amplicon. Therefore, in some embodiments two detection polynucleotides are hybridized to each strand of an amplicon, which further increases the sensitivity of the detection method. Thus in various embodiments, an amplicon may contain virtually any number of sequences suitable for hybridizing to the detection polynucleotides, as disclosed herein.

The various polynucleotides described herein may be of any chemical composition that is suitable for the polynucleotide to carry out its intended function. Thus, in one non-limiting example, a flap probe may be of any chemical composition suitable for hybridizing to a target polynucleotide and for providing a flap sequence suitable for release by the 5′-3′ nuclease activity of a polymerase under the conditions of the disclosed methods. Therefore, in some embodiments, a flap probe may comprise nucleobases that are substantially resistant to the 5′-3′ nuclease activity of a polymerase with the exception of a sequence within the probe that is to be cleaved by the nuclease activity. In another non-limiting example, a primer may be of any chemical composition suitable for hybridizing to a template and for providing a substrate for template directed primer extension by the action of a polymerase. In another non-limiting example, a ligation probe may be of any chemical composition suitable for hybridizing to a amplicon and being ligated by a thermostable ligase. Determining the types of nucleobase polymers suitable for the function of each polynucleotide is within the abilities of the skilled artisan.

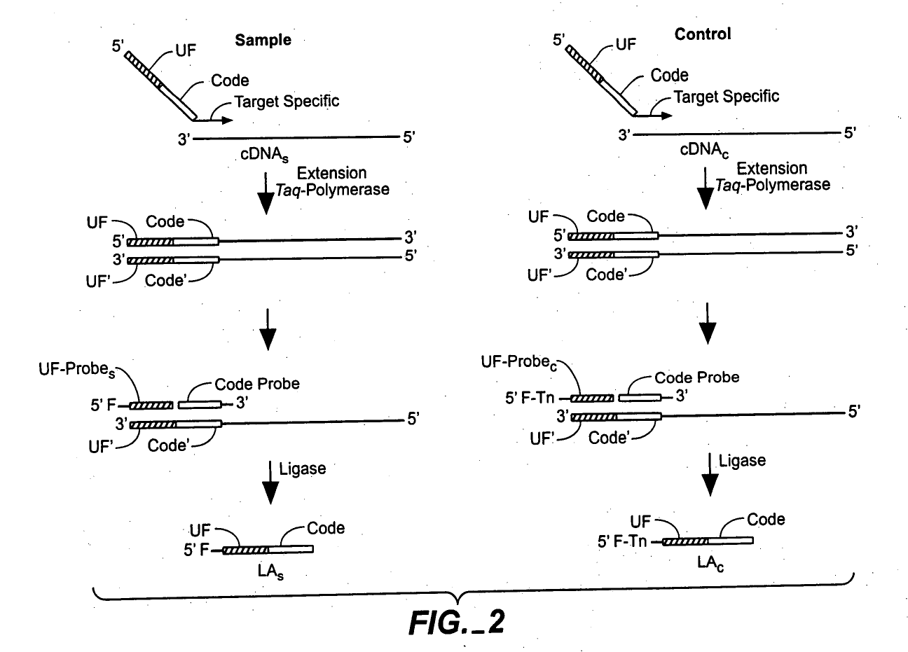

Therefore, by “nucleobase” is meant naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases disclosed in FIGS. 2(A) and 2(B) of Buchardt et al. (U.S. Pat. No. 6,357,163, WO 92/20702 and WO 92/20703).

Nucleobases can be linked to other moieties to form nucleosides, nucleotides, and nucleoside/tide analogs. As used herein, “nucleoside” refers to a compound consisting of a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. When the nucleoside base is purine or 7-deazapurine, the pentose is attached at the 9-position of the purine or deazapurine, and when the nucleoside base is pyrimidine, the pentose is attached at the 1-position of the pyrimidine (see, e.g., Komberg and Baker, DNA Replication, 2nd Ed. (W. H. Freeman & Co. 1992)). The term “nucleotide” as used herein refers to a phosphate ester of a nucleoside, e.g., a mono-, a di- , or a triphosphate ester, wherein the most common site of esterification is the hydroxyl group attached to the C-5 position of the pentose. “Nucleotide 5′-triphosphate” refers to a nucleotide with a triphosphate ester group at the 5′ position. The term “nucleoside/tide” as used herein refers to a set of compounds including both nucleosides and/or nucleotides.

“Nucleobase polymer or oligomer” refers to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotide or oligonucleotide” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

In some embodiments, a nucleobase polymer is an polynucleotide analog or an oligonucleotide analog. By “polynucleotide analog or oligonucleotide analog” is meant nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani, 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190.

In some embodiments, a nucleobase polymer is a polynucleotide mimic or oligonucleotide mimic. By “polynucleotide mimic or oligonucleotide mimic” is meant refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 98/03542 (see also, Haaima et al., 1996, Angewandte Chemie Int'l Ed. in English 35:1939-1942; Lesnick et al., 1997, Nucleosid. Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. No. 5,698,685, U.S. Pat. No. 5,470,974, U.S. Pat. No. 5,378,841 and U.S. Pat. No. 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g., Stirchak and Summerton, 1987, J. Org. Chem. 52:4202); amide backbones (see, e.g., Lebreton, 1994, Synlett. February, 1994:137); methylhydroxyl amine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide nucleic acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4:1081-1082; Petersen et al., 1996, Bioorganic & Medicinal Chemistry Letters, 6:793-796; Diderichsen et al., 1996, Tett. Lett. 37:475-478; Fujii et al., 1997, Bioorg. Med. Chem. Lett. 7:637-627; Jordan et al., 1997, Bioorg. Med. Chem. Lett. 7:687-690; Krotz et al., 1995, Tett. Lett. 36:6941-6944; Lagriffoul et al., 1994, Bioorg. Med. Chem. Lett. 4:1081-1082; Diederichsen, 1997, Bioorg. Med. Chem. 25 Letters, 7:1743-1746; Lowe et al., 1997, J. Chem. Soc. Perkin Trans. 1, 1:539-546; Lowe et al., 1997, J. Chem. Soc. Perkin Trans. 11:547-554; Lowe et al., 1997, 1. Chem. Soc. Perkin Trans. 1 1:555-560; Howarth et al., 1997, I. Org. Chem. 62:5441-5450; Altmann et al., 1997, Bioorg. Med. Chem. Lett., 7:1119-1122; Diederichsen, 1998, Bioorg. Med. Chem. Lett., 8:165-168; Diederichsen et al., 1998, Angew. Chem. mt. Ed., 37:302-305; Cantin et al., 1997, Tett. Lett., 38:4211-4214; Ciapetti et al., 1997, Tetrahedron, 53:1167-1176; Lagriffoule et al., 1997, Chem. Eur. 1.′ 3:912-919; Kumar et al., 2001, Organic Letters 3(9):1269-1272; and the Peptide-Based Nucleic Acid Mimics (PENAMs) of Shah et al. as disclosed in WO 96/04000.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500).

In some embodiments, a nucleobase polymer is a chimeric oligonucleotide. By “chimeric oligonucleotide” is meant a nucleobase polymer or oligomer comprising a plurality of different polynucleotides, polynucleotide analogs and polynucleotide mimics. For example a chimeric oligo may comprise a sequence of DNA linked to a sequence of RNA. Other examples of chimeric oligonucleotides include a sequence of DNA linked to a sequence of PNA, and a sequence of RNA linked to a sequence of PNA.

In some embodiments, a polynucleotide (e.g., an amplification primer, a detection polynucleotide) comprises one or more non-nucleobase moieties. Non-limiting examples of non-nucleobase moieties include but are not limited to a ligand, as described above, a “blocking moiety” suitable for inhibiting polymerase extension of the ₃′ terminus of a probe when it is hybridized to a target sequence, and moieties suitable for producing a detectable signal. “Detectable moiety,” “detection moiety” or “label” refer to a moiety that, when attached to the disclosed polynucleotides and other compositions, render such compositions detectable or identifiable using known detection systems (e.g., spectroscopic, radioactive, enzymatic, chemical, photochemical, biochemical, immunochemical, chromatographic or electrophoretic systems). Non-limiting examples of labels include isotopic labels (e.g., radioactive or heavy isotopes), magnetic labels; spin labels, electric labels; thermal labels; colored labels (e.g., chromophores), luminescent labels (e.g., fluorescers, chemiluminescers), enzyme labels (e.g., horseradish peroxidase, alkaline phosphatase, luciferase, β-galactosidase) (Ichiki, et al.,1993, J. Immunol. 150(12):5408-5417; Nolan, et al., 1988, Proc. Natl. Acad. Sci. USA 85(8):2603-2607)), antibody labels, chemically modifiable labels, and mobility modifier labels. In addition, in some embodiments, such labels include components of ligand-binding partner pairs, as described above.

“Fluorescent label,” “fluorescent moiety,” and “fluorophore” refer to a molecule that may be detected via its inherent fluorescent properties. Examples of suitable fluorescent labels include, but are not limited to, fluorescein, rhodamine, tetramethylrhodamine, eosin, erythrosin, coumarin, methyl-coumarins, pyrene, Malacite Green, stilbene, Lucifer Yellow, Cascade BlueJ, Texas Red, IAEDANS, EDANS, BODIPY FL, LC Red 640, phycoerythrin, LC Red 705, Oregon green, Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE), FITC, Rhodamine, Texas Red (Pierce, Rockford, Ill.), Cy5, Cy5.5, Cy7 (Amersham Life Science, Pittsburgh, Pa.) and tandem conjugates, such as but not limited to, Cy5PE, Cy5.5PE, Cy7PE, Cy5.5APC, Cy7APC. In some embodiments, suitable fluorescent labels also include, but are not limited to, green fluorescent protein (GFP; Chalfie, et al., 1994, Science 263(5148):802-805), EGFP (Clontech Laboratories, Inc., Palo Alto, Calif.), blue fluorescent protein (BFP; Quantum Biotechnologies, Inc. Montreal, Canada; Heim et al, 1996, Curr. Biol. 6:178-182; Stauber, 1998, Biotechniques 24(3):462-471;), enhanced yellow fluorescent protein (EYFP; Clontech Laboratories, Inc., Palo Alto, Calif.), and renilla (WO 92/15673; WO 95/07463; WO 98/14605; WO 98/26277; WO 99/49019; U.S. Pat. Nos. 5,292,658, 5,418,155, 5,683,888, 5,741,668, 5,777,079, 5,804,387, 5,874,304, 5,876,995 and U.S. Pat. No. 5,925,558). Further examples of fluorescent labels are found in Haugland, Handbook of Fluorescent Probes and Research, 9^{th} Edition, Molecule Probes, Inc. Eugene, Oreg. (ISBN 0-9710636-0-5).

In other embodiments, a fluorescent moiety may be an acceptor or donor molecule of a fluorescence energy transfer (FET) or fluorescent resonance energy transfer (FRET) system, which utilize distance-dependent interactions between the excited states of two molecules in which excitation energy is transferred from a donor molecule to an acceptor molecule (see Bustin, 2000, J. Mol. Endocrinol. 25:169-193; WO2004003510). As known in the art, these systems are suitable for detecting or monitoring changes in molecular proximity, including but not limited to, the release of the “flap” sequence by the 5′-3′ nuclease activity of a polymerase. Therefore, in some embodiments, a flap probe is labeled with donor and acceptor moieties, which provide a detection system suitable for monitoring the release of the flap sequence in real-time. In some embodiments, the transfer of energy from donor to acceptor results in the production of a detectable signal by the acceptor. In another embodiment, the transfer of energy from donor to acceptor results in quenching of the fluorescent signal produced by the donor. Thus, to detect or monitor the release of a “flap” sequence, the “flap” sequence and the target specific sequence of a flap probe each comprise a donor or acceptor moiety in energy transfer proximity. Therefore, depending upon the type of donor-acceptor moieties utilized, the release of the “flap” sequence may be detected or monitored by an increase or decrease in fluorescence signal. In some embodiments, the ligation of ligation probes may be monitored in an analogous fashion. Examples of donor-acceptor pairs suitable for producing a fluorescent signal include but are not limited to fluorescein-tetramethylrhodamine, IAEDANS-fluorescein, EDANS-dabcyl, fluorescein-QSY 7, and fluorescein-QSY 9. Examples of donor-acceptor pairs suitable for quenching a fluorescent signal include but are not limited to FAM-DABCYL, HEX-DABCYL, TET-DABCYL, Cy3-DABCYL, Cy5-DABCYL, Cy5.5-DABCYL, rhodamine-DABCYL, TAMRA-DABCYL, JOE-DABCYL, ROX-DABCYL, Cascade Blue-DABCYL, Bodipy-DABCYL, FAM-MGB, Vic-MGB, Ned-MGB, ROX-MGB.

In some embodiments, a label is an mobility modifier. “Mobility modifier” refers to a moiety capable of producing a particular mobility in a mobility-dependent analysis technique, such as, electrophoresis (see, e.g., U.S. Pat. Nos. 5,470,705, 5,514,543, 6,395,486 and 6,734,296). Thus, in some embodiments, a mobility modifier is an electrophoresis mobility modifier. In some embodiments, an electrophoresis mobility modifier can be a polynucleotide polymer (e.g., a ligation partner). In some embodiments, an electrophoresis mobility modifier can be a nonpolynucleotide polymer. Various non-limiting examples of non-polynucleotide electrophoresis mobility modifiers include but are not limited to polyethylene oxide, polyglycolic acid, polylactic acid, polypeptide, oligosaccharide, polyurethane, polyamide, polysulfonamide, polysulfoxide, polyphosphonate, and block copolymers thereof, including polymers composed of units of multiple subunits linked by charged or uncharged linking groups.

The use of detectable moieties in the detection of specific nucleotides at selected positions of a target sequence by the disclosed methods is within the abilities of the skilled artisan. Factors to be considered in selecting the number and types of detectable moieties and their distribution among the various polynucleotides, include but are not limited to, the number of target polynucleotides to be analyzed (e.g., single-plex vs. multiplex analysis), the method selected for detecting the modified products of the detection polynucleotides, the number and types of detectable moieties than may be discriminated, and the extent to which each specific nucleotide is to be discriminated. For example, in some embodiments, flap sequences may comprise detectable moieties. In some embodiments, such as multiplex target sequence analysis, each flap sequence may comprise a detectable moiety that may be discriminated from the detectable moieties of other flap sequences. Therefore, each released flap sequence may be identified by the emission of a unique signal. However, in some embodiments, each flap sequence may comprise the identical detectable moiety. In these embodiments, each released flap sequence may be individually discriminated if, for example, each flap sequence is substantially unique. For example, in embodiments in which each flap sequence differs in length by at least one nucleobase, the individual flap sequence may be conveniently discriminated by capillary electrophoresis. However, in embodiments in which each flap sequence comprises an identical detectable moiety and comprises a sequence of identical length, the individual flap sequences may be discriminated if, for example, the each flap sequence does not share statistically significant sequence homology with the other flap sequences. Therefore, in this embodiment, each released flap sequence may be ligated to a unique ligation partner each comprising a distinguishable electrophoresis mobility modifier to form distinguishable ligation amplicons, which also may be individually detected by capillary electrophoresis (e.g., ABI Prism® capillary electrophoresis instruments, Applied Biosystems, Foster City, Calif.). As the skilled artisan will appreciate, these examples of approaches to discriminate individual flap sequence also may be applied to the discrimination of individual ligation amplicons.

In various embodiments, a modified detection probe, can be monitored in real-time by carrying out the disclosed methods in the presence of a reporter molecule that generates a detectable signal proportion to the amount of product present in a reaction. By “reporter molecule” herein is meant a molecule that produces a differential signal when specifically or non-specifically bound to a single-stranded polynucleotide relative to the unbound molecule. Non-limiting examples of reporter molecules include sequence-independent binding agents and sequence-specific binding agents. By “sequence-independent binding” is meant differential binding that is based on structure other than the sequence of a polynucleotide. Therefore, non-limiting examples of structure-specific binding agents include intercalating agents, such as, actinomycin D which fluoresces red when bound to single-stranded polynucleotides and green when bound to double-stranded polynucleotides. By “sequence-specific binding” is meant differential binding based on the sequence of a polynucleotide. Therefore, in some embodiments, a sequence-specific reporter molecule is an oligonucleotide probe. Such oligonucleotide probes include, but are not limited to, hydrolyzable probes (see, e.g., 5′-nuclease probes, (e.g., self-quenching fluorescent probes, e.g., TaqMan® probes), various stem-loop molecular beacons (see, e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA molecular beacons (see, e.g., U.S. Pat. No. 6,355,421), linear PNA beacons (see, e.g. Kubista et al., 2001, SPIE 4264:53-58), non-FRET probes (see, e.g., U.S. Pat. No. 6,150,097) and the various different sunrise primers, scorpion probes, cyclicons (Kandimalla et al., 2000, Bioorg Med Chem. 8(8):1911-6), peptide nucleic acid (PNA) light-up probes, self-assembled nanoparticle probes (Taton et al., 2000, Science. 289(5485): 1757-60), dual-probe systems, and ferrocene-modified probes described, for example, in U.S. Pat. No. 6,485,901; Mhlanga et al., 2001, Methods. 25:463-471; Whitcombe et al., 1999, Nat. Biotechnol. 17:804-807; Isacsson et a., 2000, Mol Cell Probes. 14:321-328; Svanvik et al., 2000, Anal. Biochem. 281:26-35; Wolffs et a., 2001, Biotechniques. 766:769-771; Tsourkas et al., 2002, Nucleic Acids Res. 30:4208-4215; Riccelli et al., 2002, Nucleic Acids Res. 30:4088-4093; Zhang et al., 2002, Shanghai. 34:329-332; Maxwell et al., 2002, J Am Chem Soc. 124:9606-9612; Broude et al., 2002, Trends Biotechnol. 20:249-56; Huang et al., 2002, Chem Res Toxicol. 15:118-126; and Yu et al., 2001, J. Am. Chem. Soc. 14:11155-11161) and hydrolyzable “flap” probes, as described above and in U.S. Patent Application Ser. Nos. 60/584,621; 60/584,665; 60/584,643, each filed Jun. 30, 2004.

In some embodiments, the detectable signal is measured at one or more discrete time points or is continuously monitored in real-time. In these embodiments, continuous or discrete monitoring may utilize a reporter molecule comprising a donor-acceptor pair, e.g., fluorophore-quencher pair, as described above. Detection of the fluorescent signal can be performed in any appropriate way based, in part, upon the type of reporter molecule employed (e.g., 5′-nuclease probe vs. a molecular beacon) as known in the art. In some embodiments, the signal may be compared against a control signal or standard curve. Non-limiting examples of existing apparatuses that may be used to monitor the reaction in real-time or take one or more single time point measurements include, Models 7300, 7500, and 7700 Real-Time PCR Systems (Applied Biosystems, Foster City, Calif.); the MyCyler and icycler Thermal Cyclers (Bio-Rad, Hercules, Calif.); the Mx3000P™ and Mx4000® (Stratagene®, La Jolla, Calif.); the Chromo 4™ Four-Color Real-Time System (MJ Research, Inc., Reno, Nev.); and the LightCycler® 2.0 Instrument (Roche Applied Science, Indianapolis, Ind.).

Also provided are kits for use in practicing the various embodiments of the disclosed methods. Therefore, in some embodiments kits include one or more sets of amplification primers for producing one more amplicons and detection polynucleotides suitable for detecting the one or more amplicons. In some embodiments, the amplification primers comprise sequences, including but not limited to, one or more universal sequences and/or code sequences, which in some embodiments provide hybridization targets for the detection polynucleotides. In some embodiments, the detection polynucleotides comprise one or more primers and flap probes. In some embodiments, the detection polynucleotides comprises two or more ligation probes. In some embodiments, a kit may further comprise a polymerase suitable to amplify a target sequence and/or a polymerase having 5′-3′ nuclease activity. In various embodiments, kits may further comprise moieties suitable for producing a detectable signal or reporter molecules suitable for monitoring, for example, the accumulation of the target sequence or modification of a detection polynucleotide, as described above.

The following examples are offered by way of illustration and not by way of limitation. All literature and similar materials cited in this application, including but not limited to, patents, patent applications, articles, books, and treatises, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

### 6. EXAMPLES

### Example 1

### Gene Expression Analysis of Liver cDNA

Liver cells were analyzed for the presence of mRNA transcripts encoding apolipoprotein C2 (APOC2), protein phosphatase 1 (PPP1CA), human tissue plasminogen activator (PLAT), peroxisomal PTS2 receptor (PEX7), copper efflux transporter (ATP7A), protein kinase C alpha (PRKCA), breast cancer susceptibility protein (BRCA1), ventricular myosin regulatory light chain (MYL2), ATP synthase beta subunit (ATP5B), cholesteryl ester transfer protein (CETP), eukaryotic translation initiation factor 1A (EIF1A), and cytochrome c oxidase (COX6b). Liver mRNA was reverse transcribed and cDNA was amplified by PCR in a multiplex format using a pair of forward and reverse primers for each target sequence. Each forward primer comprised a 5′ forward universal sequence, a code sequence, and a 3′ target specific sequence (Table 2, e.g., FP-Code-1-APOC2). Each reverse primer comprised a 5′ reverse universal sequence and a 3′ target specific sequence (Table 2, e.g., UR-APOC2). The double-stranded amplicons were further amplified using universal forward (Table 2, UF) and universal reverse primers (Table 2, UR).

One strand of the amplicons was hybridized to two ligation probes. The first probe was amplicon-specific (Table 2, e.g., LP-Code-1-APOC2) and comprised the code sequence of one of the forward primers. Each amplicon-specific probe also comprised a 3′-poly(T) tail of variable length suitable to distinguish each of the amplicon-specific probes. The second probe comprised the universal forward sequence and the fluorescent label, FAM (Table 2, U-LP). The two probes were hybridized to one strand of the amplicons, ligated to produce ligation amplicons, which were detected by capillary electrophoresis.

Liver cDNA was amplified in a reaction comprising 1× ABI PCR Mix, 10 nM each forward and reverse primer, 1 μM each universal forward and reverse primer and Ampli-Taq® GOLD (Applied Biosystemrs, Foster City, Calif.). The quantity of liver cDNA amplified was 1 pg, 10 pg and 100 pg. The reaction was incubated at 95° C. for 10 min., thermocycled 40× (95° C. for 15 sec.−60 C for 1 min.), incubated at 99.9° C. for 30 min, and held at 4° C.

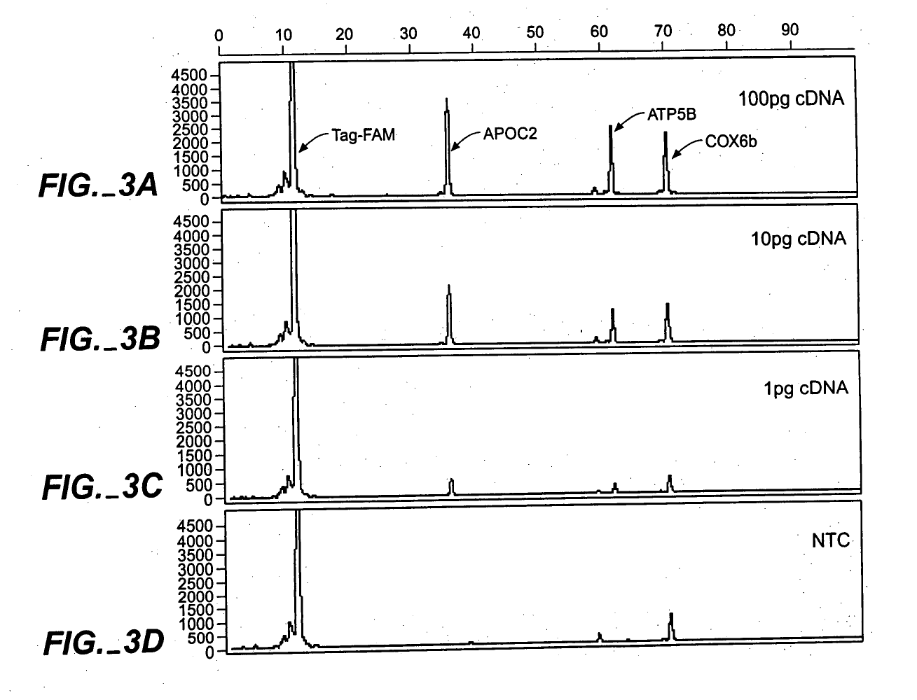

Ligation amplicons were produced in a reaction comprising 0.5 μl of each amplification reaction, 1× ABI Ligase Buffer, 20 nM each amplicon-specific ligation probe, 100 nM universal probe, and 0.5 UI/μl AK16D. The ligation reaction was incubated at 94° C. for 5 sec., thermocycled 30× (94° C. for 5 sec.−65 C for 1 min.), incubated at 99° C. for 10 min, and held at 4° C. The ligation amplicons were analyzed by capillary electrophoresis. The results, shown in FIG. 3A-D, indicate that mRNA transcripts encoding APOC2, ATP5B and COX6b were expressed by liver cells and that a detectable signal was produced using about 1 pg liver cDNA.

### Example 2

### Gene Expression Analysis of Human Reference cDNA (HR-cDNA)

Human Reference-cDNA was analyzed for the presence of mRNA transcripts encoding APOC2, PPP1CA, PEX7, ATP7A, BRCA1, ATP5B, CETP and EIF1A. The cDNA was amplified by PCR in a multiplex format using a pair of forward and reverse primers for each target sequence. Each forward primer comprised a 5′ forward universal sequence, a code sequence, and a 3′ target specific sequence (Table 2, e.g., FP-Code-1-APOC2). Each reverse primer comprised a 5′ reverse universal sequence and a 3′ target specific sequence (Table 2, e.g., UR-APOC2). One strand of the double-stranded amplicons was further amplified using universal reverse primer (Table 2, UR).

The strand amplified by the universal reverse primer was hybridized to two ligation probes. The first probe was amplicon-specific (Table 2, e.g., LP-Code-1-APOC2) and comprised the code sequence of one of the forward primers. Each amplicon-specific probe also comprised a 3′-poly(T) tail of variable length suitable to distinguish each of the amplicon-specific probes. The second probe comprised the universal forward sequence and the fluorescent label, FAM (Table 2, U-LP). The two probes were hybridized to one strand of the amplicons, ligated to produce ligation amplicons, which were detected by capillary electrophoresis.

HR-cDNA was amplified in a reaction comprising 1× ABI PCR Mix, 10 nM each forward primer, 5 nM each reverse primer, 1 μM universal reverse primer, an additional 1.25 U Ampli-Taq® GOLD (Applied Biosystems, Foster City, Calif.) and 250 ng human sperm DNA (HS-DNA). The quantity of HR cDNA amplified was 100 pg, 10 pg and 1 pg. No HR cDNA was added to a negative control. In control reactions, 10 fM, 1 fM and 100 aM of cloned CETP, ATP7B, BRCA1 and PEX7 DNA were amplified. All reactions were thermocycled 45× (95° C. for 15 sec.−65 C for I min.).

Ligation amplicons were produced in a reaction comprising 0.5 μl of each amplification reaction, 1× ABI Ligase Buffer, 20 nM each amplicon-specific ligation probe, 100 nM universal probe, and 0.5 UI/μl AK16D. The ligation reaction was incubated at 94° C. for 5 sec., thermocycled 30× (94° C. for 5 sec.−65 C for 1 min.), incubated at 99° C. for 10 min, and held at 4° C.

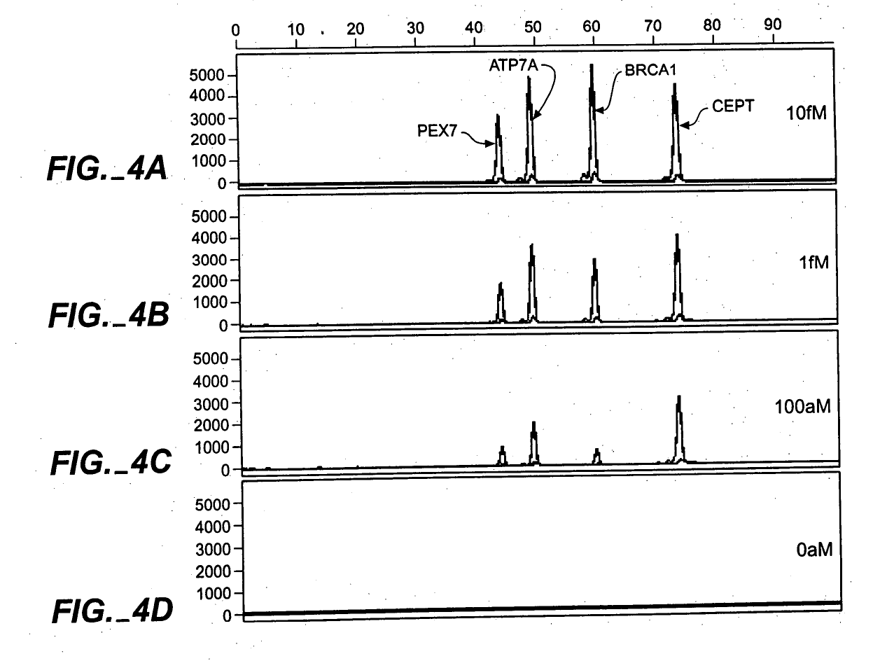

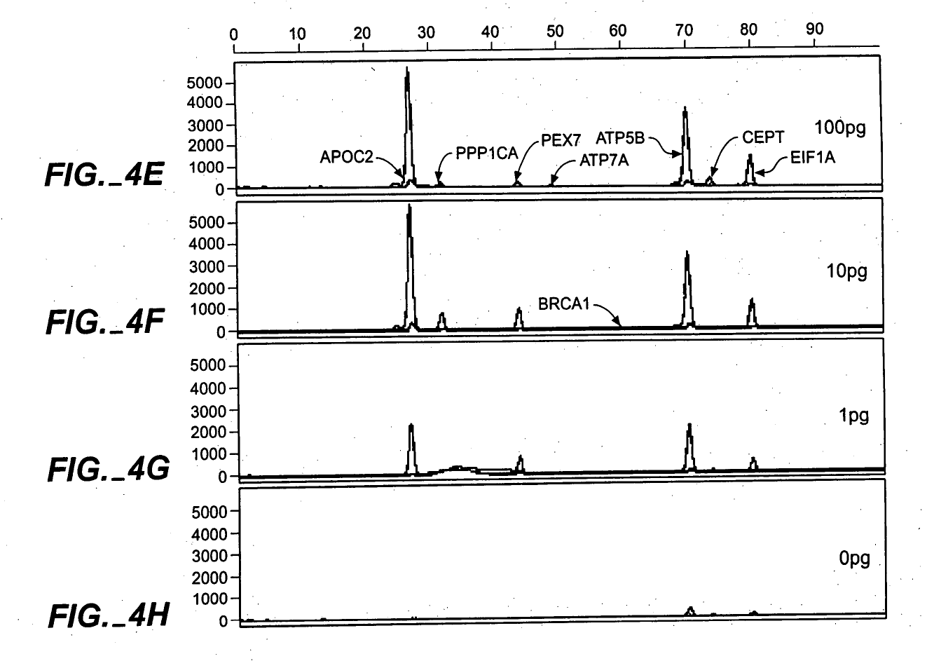

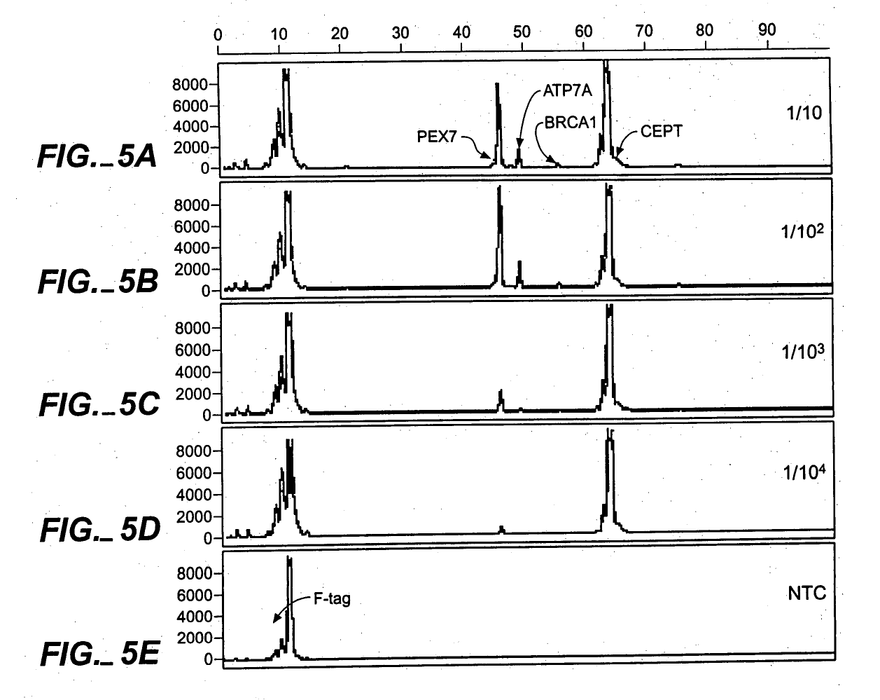

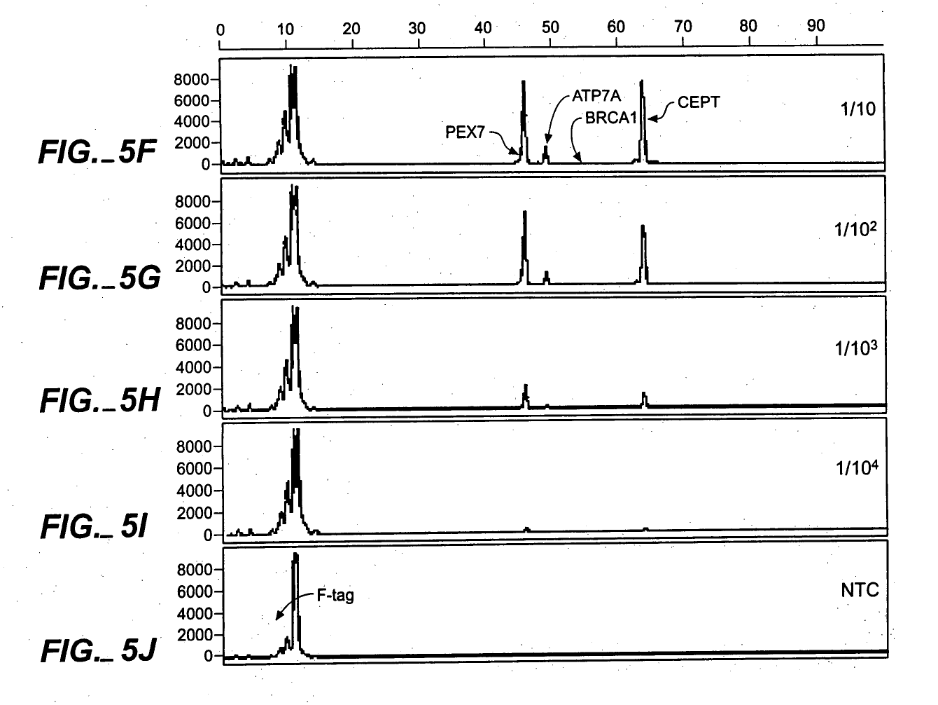

Ligation amplicons were analyzed by capillary electrophoresis. The results for the control reactions are shown in FIGS. 4A-D and indicate a sensitivity of at least about 100 aM. The results for the HR-cDNA are shown in FIGS. 4E-H and indicate that APOC2, ATP5B and EIF1A are the highest expressed genes analyzed. Other genes expressed at very low but detectable amounts were PPP1CA, PEX7, CETP.

### Example 3

### Sensitivity and Dynamic Range of an Embodiment of Gene Expression Analysis

Various concentrations of cloned ATP7A, BRCA1, PEX7 and CETP cloned DNA were analyzed in a multiplex reaction similar to the method described in Example 2. The DNA was amplified in a reaction comprising 1× ABI PCR Mix, 5 nM each forward and reverse primer, 5 nM each reverse primer, and 1 μM universal reverse primer. The various amounts of DNA analyzed per reaction is shown in Table 1. The reactions were thermocycled 50× (95° C. for 15 sec.−65° C. for 1 min. The ligation reaction was performed as described in Example 2.

TABLE 1

| 10. | 9. | 8. | | 7. | | 6. | | 5. | | 4. | | 3. | | 2 | | 1 | | |
|---|---|---|---|---|---|---|---|---|---|---|---|---|---|---|---|---|---|---|
| 0 | 0 | 0.01 | fM | 0.1 | fM | 1 | fM | 10 | fM | 0.01 | fM | 0.1 | fM | 1 | fM | 10 | fM | ATP7A |
| 0 | 0 | 0.001 | fM | 0.01 | fM | 0.1 | fM | 1 | fM | 0.01 | aM | 0.1 | aM | 1 | aM | 10 | aM | BRCA1 |
| 0 | 0 | 0.01 | pM | 0.1 | pM | 1 | pM | 10 | pM | 0.1 | fM | 1 | fM | 10 | fM | 100 | fM | CETP |
| 0 | 0 | 0.1 | fM | 1 | fM | 10 | fM | 100 | fM | 0.1 | fM | 1 | fM | 10 | fM | 100 | fM | PEX7 |

Results

The results indicate that variable amounts of each sequence may be detected by the disclosed methods and that the signal produced is proportional to the amount or concentration of target sequence.

### Example 4

### Gene Expression Analysis of Liver and Brain cDNA

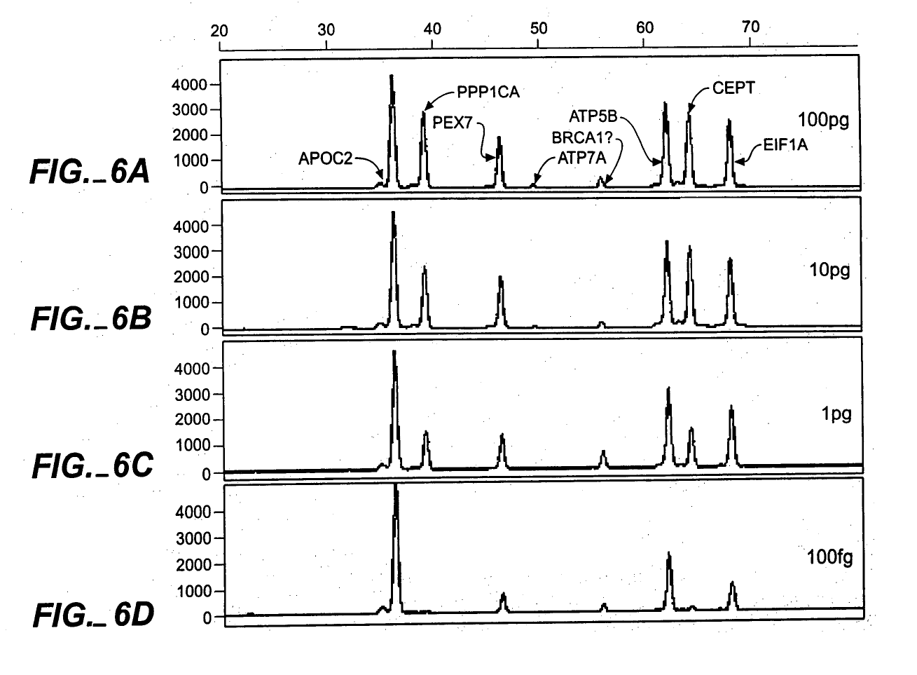

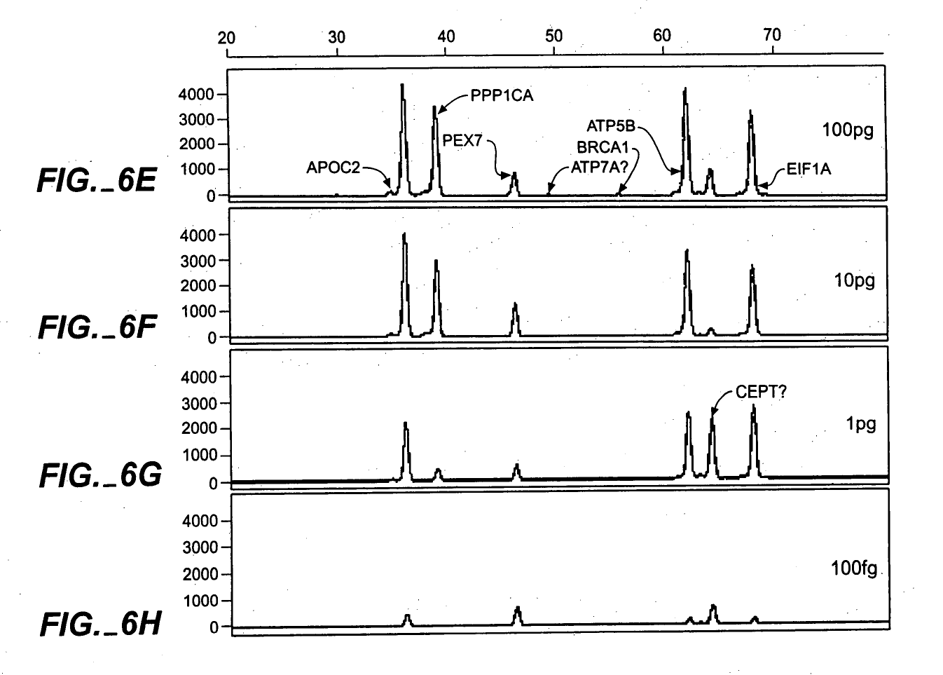

Liver and brain cDNA were analyzed for sequences encoding APOC2, PPP1CA, PEX7, ATP7A, BRCA1, ATP5B, CETP and EIF1A. The amplification and ligation reactions followed the procedures described in Example 1 with the exception that HS-DNA was not included in the amplification reaction. The quantity of cDNA employed in each reaction is shown in FIGS. 6A-H. FIGS. 6A-D show the results for liver cDNA. FIGS. 6E-H show the results obtained for brain cDNA.

TABLE 2

| | | |
|---|---|---|
| Polynucleotide | Sequence 5′-3′ | Seq ID NO: |

| | | |
|---|---|---|
| FP-Code-1-APOC2 | GTGTCGTGGAGTCGGCAAGAAGCGAGCGGGAACAGGCCAACAGGCATTTTTACTGACCAAGTTCT | SEQ ID NO:01 |
| FP-Code-2-PPP1CA | GTGTCGTGGAGTCGGCAAGAGGAACACCACGCAGCGCAGGTTGTGCAGAAAAACAAGTCCTAAAGT | SEQ ID NO:02 |
| FP-Code-3-PLAT | GTGTCGTGGAGTCGGCAAGAGCAGTGCTCACCGTCCGCGACACATTGATGTCTCCTGCTGTACTAA | SEQ ID NO:03 |
| FP-Code-4-PEX7 | GTGTCGTGGAGTCGGCAAGACGGAGTGGCACCAGCGGGAATGAGTTGTGACTGGTGTAAATACAATGA | SEQ ID NO:04 |
| FP-Code-5-ATP7A | GTGTCGTGGAGTCGGCAAGAGCAGCAGGCCAAAGCGAGCGGGGAAGATGATGACACTGCATTATAA | SEQ ID NO:05 |
| FP-Code-6-PRKCA | GTGTCGTGGAGTCGGCAAGAGTCCGAGCCCTCACGCAGCGACTGATGACCCCAGGAGCAA | SEQ ID NO:06 |
| FP-Code-7-BRCA1 | GTGTCGTGGAGTCGGCAAGAGCAGGACGACGCGGGTGGAACCAAAGACAGTCTTCTAATTCCTCATT | SEQ ID NO:08 |
| FP-Code-8-MYL2 | GTGTCGTGGAGTCGGCAAGATGGCGGTCTGCTGACGGTCGGTGCTGAAGGCTGATTACGTT | SEQ ID NO:07 |
| FP-Code-9-ATP5B | GTGTCGTGOAGTCGGCAAGAGTGGGTCCCGGAAGCGTGCTCCCGTGCACGGAAAATACAG | SEQ ID NO:09 |
| FP-Code-10-CETP | GTGTCGTGGAGTCGGCAAGAGCCTCGAGCCAACACCGCCTCAGATTACACCAAAGACTGTTTCCAA | SEQ ID NO:10 |
| FP-Code-11-EIF1A | GTGTCGTGGAGTCGGCAAGATGGCCGGACAGGAGACACGCCAAGATTGGCGGCATTGG | SEQ ID NO:11 |
| FP-Code-12-COX6b | GTGTCGTGGAGTCGGCAAGAGCCTGCCTTCACGAGCCCAATGGGGCAGAGGGACTGGTA | SEQ ID NO:12 |
| UR-APOC2 | ACCGACTCCAGCTCCCGAACACTCTCCCCTTGTCCACTGATG | SEQ ID NO:13 |
| UR-ATP5B | ACCGACTCCAGCTCCCGAACCCTGTGAAGACCTCAGCAACCT | SEQ ID NO:14 |
| UR-EIF1A | ACCGACTCCAGCTCCCGAACCCCGGCCGCAGGAT | SEQ ID NO:15 |
| UR-PPP1CA | ACCGACTCCAGCTCCCGAACTGATTGGACATGACACAGGATACA | SEQ ID NO:16 |
| UR-PLAT | ACCGACTCCAGCTCCCGAACAGCCCCACTGCGGTACTG | SEQ ID NO:17 |
| UR-CETP | ACCGACTCCAGCTCCCGAACTGACTGCAGGAAGCTCTGGAT | SEQ ID NO:18 |
| UR-PEX7 | ACCGACTCCAGCTCCCGAACAAGTCCCAGCCTCTCAAACTACAG | SEQ ID NO:19 |
| UR-ATP7A | ACCGACTCCAGCTCCCGAACTGGAATGCTGTGTCAGTGCATGA | SEQ ID NO:20 |
| UR-PRKCA | ACCGACTCCAGCTCCCGAACGGTGGGGCTTCCGTAAGTGT | SEQ ID NO:21 |
| UR-BRCA1 | ACCGACTCCAGCTCCCGAACTCATGCCAGAGGTCTTATATTTTAAGAG | SEQ ID NO:22 |
| UR-MYL2 | ACCGACTCCAGCTCCCGAACCAACCTCCTCCTTGGAAAACC | SEQ ID NO:23 |
| UR-Cox6b | ACCGACTCCAGCTCCCGAACACCGCTAAAGGAGGCGATATC | SEQ ID NO:24 |
| UR | ACCGACTCCAGCTCCCGAAC | SEQ ID NO:25 |
| UF | GTGTCGTGGAGTCGGCAA | SEQ ID NO:26 |
| U-LP | Fam-TGTGTCGTGGAGTCGGCAAGA | SEQ ID NO:27 |
| LP-Code-1-APOC2 | PO₄-AGCGAGCGGGAACAGGCCAATT | SEQ ID NO:28 |
| LP-Code-2-PPP1CA | PO₄-GGAACACCACGCAGCGCAGGTTTTT | SEQ ID NO:29 |
| LP-Code-3-PLAT | PO₄-GCAGTGCTCACCGTCCGCGATTTTTTTT | SEQ ID NO:30 |
| LP-Code-4-PEX7 | PO₄-CGGAGTGGCACCAGCGGGAATTTTTTTTTTT | SEQ ID NO:31 |
| LP-Code-5-ATP7A | PO₄-GCAGCAGGCCAAAGCGAGCGTTTTTTTTTTTTTT | SEQ ID NO:32 |
| LP-Code-6-PRKCA | PO₄-GTCCGAGCCCTCACGCAGCGTTTTTTTTTTTTTTTTT | SEQ ID NO:33 |
| LP-Code-7-BRCA1 | PO₄-GCAGGACGACGCGGGTGGAATTTTTTTTTTTTTTTTTTTT | SEQ ID NO:34 |
| LP-Code-8-MYL2 | PO₄-TGGCGGTCTGCTGAGCGGTCTTTTTTTTTTTTTTTTTTTTTTT | SEQ ID NO:35 |
| LP-Code-9-ATP5B | PO₄-GTGGGTCCCGGAAGCGTGCTTTTTTTTTTTTTTTTTTTTTTTTTTT | SEQ ID NO:36 |
| LP-Code-10-CETP | PO₄-GCCTCGAGCCAACACCGCCTTTTTTTTTTTTTTTTTTTTTTTTTTTTTT | SEQ ID NO:37 |
| LP-Code-11-EIF1A | PO₄-TGGCCGGACAGGAGACACGCTTTTTTTTTTTTTTTTTTTTTTTTTTTTTTTT | SEQ ID NO:38 |
| LP-Code-12-COX6b | PO₄-GCCTGCCTTCACGAGCCCAATTTTTTTTTTTTTTTTTTTTTTTTTTTTTTTTTTT | SEQ ID NO:39 |

What is claimed is:

1. A method of analyzing a target sequence, comprising:
a) amplifying a target sequence with a polymerase, and forward and reverse amplification primers, under conditions effective to produce a double-stranded amplicon, wherein said forward primer comprises a 3′ sequence suitable for amplifying said target sequence, a code sequence, and a 5′ universal sequence, and wherein said double stranded amplicon comprises forward and reverse strands, wherein said forward strand comprises said 5′ universal sequence and said code sequence, and said reverse strand comprises sequences complementary thereto;
b) hybridizing a universal ligation probe and a code ligation probe to said reverse strand, wherein said universal ligation probe comprises said universal sequence and said code probe comprises said code sequence, and wherein the hybridizing conditions are effective for said ligation probes and said reverse strand to form a substrate for a ligase, and for said ligase to join said ligation probes to form a ligation product; and
c) detecting said ligation product.

2. The method according to claim 1, further comprising:
d) amplifying a control sequence with said polymerase, and said forward and reverse amplification primers, under conditions effective to produce a double-stranded control amplicon, wherein said forward primer comprises a 3′ sequence suitable for amplifying said control sequence, said code sequence, and said 5′ universal sequence, and wherein said control amplicon comprises forward and reverse strands, wherein said control forward strand comprises said 5′ universal sequence and said code sequence, and said control reverse strand comprises sequences complementary thereto;
e) hybridizing a control ligation probe and said code ligation probe to said reverse control strand, wherein said control ligation probe comprises said universal sequence, and wherein the hybridizing conditions are effective for said control ligation probe, said code ligation probe and said reverse control strand to form a substrate for a ligase, and for said ligase to join said control and code ligation probes to form a control ligation product;
f) detecting said control ligation product; and
g) determining therefrom the copy number of said target sequence.

3. The method according to claim 2, wherein said universal ligation probe and said control ligation probe comprise a fluorophore.

4. The method according to claim 2, wherein said universal ligation probe or said control ligation probe comprises a mobility modifier.

5. The method according to claim 2, wherein said ligation product and said control ligation product are detected by capillary electrophoresis.

6. The method according to claim 2, wherein said ligase is a thermostable ligase and said hybridizing conditions comprise multiple rounds of thermocycling.

7. The method according to claim 2, wherein the number of double-stranded amplicons and control amplicons are substantially equivalent.

8. The method according to claim 2, wherein the amplification of said target sequence and said control target sequence terminates before reaching a plateau.

9. A method of analyzing a target sequence, comprising:
a) amplifying a target sequence with a polymerase having 5′-3′ nuclease activity, and forward and reverse amplification primers, under conditions effective to produce a double-stranded amplicon, wherein said forward primer comprises a 3′ sequence suitable for amplifying said target sequence, a code sequence, and a 5′ universal sequence, and wherein said double stranded amplicon comprises forward and reverse strands, wherein said forward strand comprises said 5′ universal sequence and said code sequence, and said reverse strand comprises sequences complementary thereto;
b) hybridizing to said reverse strand a detection primer and a flap probe, wherein said detection primer comprises said universal sequence and said flap probe comprises said code sequence and a flap sequence, under conditions effective for said detection primer, flap probe and reverse strand to form a substrate for said nuclease activity and for said nuclease activity to release the flap sequence from said probe; and
c) detecting the released flap sequence.

10. The method according to claim 9, further comprising:
d) amplifying a control sequence with said polymerase, and said forward and reverse amplification primers, under conditions effective to produce a double-stranded control amplicon comprising forward and reverse strands, wherein said control forward strand comprises said 5′ universal sequence and said code sequence, and said control reverse strand comprises sequences complementary thereto;
e) hybridizing to said control reverse strand said detection primer and a control flap probe comprising a control flap sequence and said code sequence under conditions effective for said detection primer, control flap probe and control reverse strand to form a substrate for said nuclease activity and for said nuclease activity to release said control flap sequence from said control probe;
f) detecting the released control flap sequence; and
g) determining therefrom the copy number of said target sequence.

11. The method according to claim 10, where released said flap sequence and said released control flap sequence are detected by capillary electrophoresis.

12. The method according to claim 10, wherein said released flap sequence and said released control flap sequence are ligated to ligation partners to form ligation products and control ligation products, respectively.

13. The method according to claim 10, wherein said released flap sequence and said released control flap sequences comprise a fluorophore.

14. The method according to claim 10, wherein said released flap sequence or said released control flap sequence comprises a mobility modifier.

15. The method according to claim 10, wherein the number of double-stranded amplicons and double-stranded control amplicons are substantially equivalent.

16. The method according to claim 10, wherein the amplification of said target sequence and said control target sequence terminates before reaching a plateau.

17. A method of analyzing a plurality of target sequences, comprising:
a) amplifying a plurality of target sequences with a polymerase, and a plurality of forward and reverse amplification primers, under conditions effective to produce a plurality of double-stranded amplicons, wherein said forward primers comprise a 3′ sequence suitable for amplifying one of said target sequences, at least one of a plurality of code sequences, and a 5′ universal sequence, and wherein said double stranded amplicons comprise forward and reverse strands, wherein said forward strands comprise said 5′ universal sequence and one of said code sequences, and said reverse strands comprise sequences complementary thereto;
b) hybridizing a universal ligation probe and at least one of a plurality of code ligation probes to one of said reverse strands, wherein said universal ligation probe comprises said universal sequence and each code ligation probe comprises one of said code sequences, and wherein the hybridizing conditions are effective for said ligation probes and said reverse strand to form substrates for a ligase, and for said ligase to join said ligation probes to form a plurality of ligation products; and
c) detecting said ligation products.

18. The method according to claim 17, further comprising:
d) amplifying a plurality of control sequences with said polymerase, and said plurality of forward and reverse amplification primers, under conditions effective to produce a plurality of double-stranded control amplicons comprising forward and reverse strands, wherein said forward control strands comprise said 5′ universal sequence and one of said code sequences, and said reverse control strand comprises sequences complementary thereto;
e) hybridizing a control ligation probe comprising said universal sequence and at least one of said code ligation probes to at least one of said reverse control strands under conditions effective for said control ligation probes and said code probes to form substrates for a ligase, and for said ligase to join said control and code probes to form a plurality of control ligation products;
f) detecting said control ligation products; and
g) determining therefrom the copy number of said target polynucleotides.

19. The method according to claim 18, wherein said universal ligation probe and said control ligation probe comprise a fluorophore.

20. The method according to claim 18, wherein one of said universal ligation probe or said control ligation probe comprises a mobility modifier.

21. The method according to claim 18, wherein said ligation products and said control ligation products are detected by capillary electrophoresis.

22. The method according to claim 18, wherein said ligase is a thermostable ligase and said hybridizing conditions comprise multiple rounds of thermocycling.

23. The method according to claim 18, wherein the number of double-stranded amplicons and control amplicons are substantially equivalent.

24. The method according to claim 18, wherein the amplification of said target sequences and said control target sequences terminate before reaching a plateau.

25. A method of analyzing a plurality of target polynucleotides, comprising:
a) amplifying a plurality of target sequences with a polymerase having 5′-3′ nuclease activity, and a plurality of forward and reverse amplification primers, under conditions effective to produce a plurality of double-stranded amplicons, wherein said forward primers comprise a 3′ sequence suitable for amplifying one of said target sequences, one of a plurality of code sequences, and a 5′ universal sequence, and wherein said double stranded amplicons comprise forward and reverse strands, wherein said forward strands comprise said 5′ universal sequence and one of said code sequences, and said reverse strand comprises sequences complementary thereto;
b) hybridizing to said reverse strands a detection primer and one of a plurality of flap probes, wherein said detection primer comprises said universal sequence and said flap probes comprise said code sequence and one of a plurality of flap sequences, under conditions effective for said detection primer, flap probes and reverse strands to form substrates for said nuclease activity and for said nuclease activity to release the flap sequences from said probes; and
c) detecting said released flap sequences.

26. The method according to claim 25, further comprising:
d) amplifying a plurality of control sequences with said polymerase, and said plurality of forward and reverse amplification primers, under conditions effective to produce a plurality of double-stranded control amplicons comprising forward and reverse strands, wherein said control forward strands comprise said 5′ universal sequence and one of said code sequences, and said control reverse strands comprise sequences complementary thereto;
e) hybridizing to said control reverse strands said detection primer and one of a plurality of control flap probes, wherein said control flap probes comprise one of said code sequences and one of a plurality of control flap sequences, under conditions effective for said detection primer, control flap probes and control reverse strands to form substrates for said nuclease activity and for said nuclease activity to release said control flap sequences from said control probes;
f) detecting the released control flap sequences; and
g) determining therefrom the copy number of said target sequences.

27. The method according to claim 26, where said released flap sequences and said released control flap sequences are detected by capillary electrophoresis.

28. The method according to claim 26, wherein said released flap sequences and said released control flap sequences are ligated to ligation partners to form a plurality of ligation products and a plurality of control ligation products, respectively.

29. The method according to claim 26, wherein said released flap sequences and said released control flap sequences comprise a fluorophore.

30. The method according to claim 26, wherein said released flap sequenced or said released control flap sequences each comprise a mobility modifier.

31. The method according to claim 26, wherein the number of double-stranded amplicons and double-stranded control amplicons are substantially equivalent.

32. The method according to claim 26, wherein the amplification of said target sequences and said control target sequences terminate before reaching a plateau.


SEQUENCE LISTING (39 sequences)

SEQ ID NO: 1 - DNA, 65 bp, Artificial Sequence
  Description of Artificial Sequence Synthetic oligonucleotide
gtgtcgtggagtcggcaagaagcgagcgggaacaggccaacaggcatttttactgaccaagttct

SEQ ID NO: 2 - DNA, 66 bp, Artificial Sequence
  Description of Artificial Sequence Synthetic oligonucleotide
gtgtcgtggagtcggcaagaggaacaccacgcagcgcaggttgtgcagaaaaacaagtcctaaagt

SEQ ID NO: 3 - DNA, 66 bp, Artificial Sequence
  Description of Artificial Sequence Synthetic oligonucleotide
gtgtcgtggagtcggcaagagcagtgctcaccgtccgcgacacattgatgtctcctgctgtactaa

SEQ ID NO: 4 - DNA, 68 bp, Artificial Sequence
  Description of Artificial Sequence Synthetic oligonucleotide
gtgtcgtggagtcggcaagacggagtggcaccagcgggaatgagttgtgactggtgtaaatacaatga

SEQ ID NO: 5 - DNA, 66 bp, Artificial Sequence
  Description of Artificial Sequence Synthetic oligonucleotide
gtgtcgtggagtcggcaagagcagcaggccaaagcgagcggggaagatgatgacactgcattataa

SEQ ID NO: 6 - DNA, 60 bp, Artificial Sequence
  Description of Artificial Sequence Synthetic oligonucleotide
gtgtcgtggagtcggcaagagtccgagccctcacgcagcgactgatgaccccaggagcaa

SEQ ID NO: 7 - DNA, 61 bp, Artificial Sequence
  Description of Artificial Sequence Synthetic oligonucleotide
gtgtcgtggagtcggcaagatggcggtctgctgacggtcggtgctgaaggctgattacgtt

SEQ ID NO: 8 - DNA, 67 bp, Artificial Sequence
  Description of Artificial Sequence Synthetic oligonucleotide
gtgtcgtggagtcggcaagagcaggacgacgcgggtggaaccaaagacagtcttctaattcctcatt

SEQ ID NO: 9 - DNA, 60 bp, Artificial Sequence
  Description of Artificial Sequence Synthetic oligonucleotide
gtgtcgtggagtcggcaagagtgggtcccggaagcgtgctcccgtgcacggaaaatacag

SEQ ID NO: 10 - DNA, 66 bp, Artificial Sequence
  Description of Artificial Sequence Synthetic oligonucleotide
gtgtcgtggagtcggcaagagcctcgagccaacaccgcctcagattacaccaaagactgtttccaa

SEQ ID NO: 11 - DNA, 58 bp, Artificial Sequence
  Description of Artificial Sequence Synthetic oligonucleotide
gtgtcgtggagtcggcaagatggccggacaggagacacgccaagattggcggcattgg

SEQ ID NO: 12 - DNA, 59 bp, Artificial Sequence
  Description of Artificial Sequence Synthetic oligonucleotide
gtgtcgtggagtcggcaagagcctgccttcacgagcccaatggggcagagggactggta

SEQ ID NO: 13 - DNA, 42 bp, Artificial Sequence
  Description of Artificial Sequence Synthetic oligonucleotide
accgactccagctcccgaacactctccccttgtccactgatg

SEQ ID NO: 14 - DNA, 42 bp, Artificial Sequence
  Description of Artificial Sequence Synthetic oligonucleotide
accgactccagctcccgaaccctgtgaagacctcagcaacct

SEQ ID NO: 15 - DNA, 34 bp, Artificial Sequence
  Description of Artificial Sequence Synthetic oligonucleotide
accgactccagctcccgaaccccggccgcaggat

SEQ ID NO: 16 - DNA, 44 bp, Artificial Sequence
  Description of Artificial Sequence Synthetic oligonucleotide
accgactccagctcccgaactgattggacatgacacaggataca

SEQ ID NO: 17 - DNA, 38 bp, Artificial Sequence
  Description of Artificial Sequence Synthetic oligonucleotide
accgactccagctcccgaacagccccactgcggtactg

SEQ ID NO: 18 - DNA, 41 bp, Artificial Sequence
  Description of Artificial Sequence Synthetic oligonucleotide
accgactccagctcccgaactgactgcaggaagctctggat

SEQ ID NO: 19 - DNA, 44 bp, Artificial Sequence
  Description of Artificial Sequence Synthetic oligonucleotide
accgactccagctcccgaacaagtcccagcctctcaaactacag

SEQ ID NO: 20 - DNA, 43 bp, Artificial Sequence
  Description of Artificial Sequence Synthetic oligonucleotide
accgactccagctcccgaactggaatgctgtgtcagtgcatga

SEQ ID NO: 21 - DNA, 40 bp, Artificial Sequence
  Description of Artificial Sequence Synthetic oligonucleotide
accgactccagctcccgaacggtggggcttccgtaagtgt

SEQ ID NO: 22 - DNA, 48 bp, Artificial Sequence
  Description of Artificial Sequence Synthetic oligonucleotide
accgactccagctcccgaactcatgccagaggtcttatattttaagag

SEQ ID NO: 23 - DNA, 41 bp, Artificial Sequence
  Description of Artificial Sequence Synthetic oligonucleotide
accgactccagctcccgaaccaacctcctccttggaaaacc

SEQ ID NO: 24 - DNA, 41 bp, Artificial Sequence
  Description of Artificial Sequence Synthetic oligonucleotide
accgactccagctcccgaacaccgctaaaggaggcgatatc

SEQ ID NO: 25 - DNA, 20 bp, Artificial Sequence
  Description of Artificial Sequence Synthetic oligonucleotide
accgactccagctcccgaac

SEQ ID NO: 26 - DNA, 18 bp, Artificial Sequence
  Description of Artificial Sequence Synthetic oligonucleotide
gtgtcgtggagtcggcaa

SEQ ID NO: 27 - DNA, 21 bp, Artificial Sequence
  Description of Artificial Sequence Synthetic oligonucleotide
tgtgtcgtggagtcggcaaga

SEQ ID NO: 28 - DNA, 22 bp, Artificial Sequence
  Description of Artificial Sequence Synthetic oligonucleotide
agcgagcgggaacaggccaatt

SEQ ID NO: 29 - DNA, 25 bp, Artificial Sequence
  Description of Artificial Sequence Synthetic oligonucleotide
ggaacaccacgcagcgcaggttttt

SEQ ID NO: 30 - DNA, 28 bp, Artificial Sequence
  Description of Artificial Sequence Synthetic oligonucleotide
gcagtgctcaccgtccgcgatttttttt

SEQ ID NO: 31 - DNA, 31 bp, Artificial Sequence
  Description of Artificial Sequence Synthetic oligonucleotide
cggagtggcaccagcgggaattttttttttt

SEQ ID NO: 32 - DNA, 34 bp, Artificial Sequence
  Description of Artificial Sequence Synthetic oligonucleotide
gcagcaggccaaagcgagcgtttttttttttttt

SEQ ID NO: 33 - DNA, 37 bp, Artificial Sequence
  Description of Artificial Sequence Synthetic oligonucleotide
gtccgagccctcacgcagcgttttttttttttttttt

SEQ ID NO: 34 - DNA, 40 bp, Artificial Sequence
  Description of Artificial Sequence Synthetic oligonucleotide
gcaggacgacgcgggtggaatttttttttttttttttttt

SEQ ID NO: 35 - DNA, 43 bp, Artificial Sequence
  Description of Artificial Sequence Synthetic oligonucleotide
tggcggtctgctgagcggtcttttttttttttttttttttttt

SEQ ID NO: 36 - DNA, 46 bp, Artificial Sequence
  Description of Artificial Sequence Synthetic oligonucleotide
gtgggtcccggaagcgtgcttttttttttttttttttttttttttt

SEQ ID NO: 37 - DNA, 49 bp, Artificial Sequence
  Description of Artificial Sequence Synthetic oligonucleotide
gcctcgagccaacaccgcctttttttttttttttttttttttttttttt

SEQ ID NO: 38 - DNA, 52 bp, Artificial Sequence
  Description of Artificial Sequence Synthetic oligonucleotide
tggccggacaggagacacgctttttttttttttttttttttttttttttttt

SEQ ID NO: 39 - DNA, 55 bp, Artificial Sequence
  Description of Artificial Sequence Synthetic oligonucleotide
gcctgccttcacgagcccaattttttttttttttttttttttttttttttttttt